SEQUENCE LISTING (3 sequences)

SEQ ID NO: 1 - protein, 42 aa, artificial
  synthetic sequence
XXHGXFXXXXXXXXXXXXXXXFXXWXXXXXXXXXXXXXXXXX

SEQ ID NO: 2 - protein, 39 aa, artificial
  synthetic sequence
HXHGXFXXXXXXXXXXXAXXXFIXWLXXXXXXXXXXXXX

SEQ ID NO: 3 - protein, 39 aa, artificial
  synthetic sequence
HXHGTFTSDXSXXXEXXAXXXFIXWLXXGXXXXXXXXXX

(19) United States
(12) Patent Application Publication
(10) Pub. No.: US 2009/0062192 A1
(43) Pub. Date: Mar. 5, 2009

(54) Dimeric Peptide Agonists of the Glp-1 Receptor

(75) Inventors: Leif Christensen, Roskilde (DK); Jesper Lau, Farum (DK); Jane Spetzler, Bronshoj (DK); Thomas Kruse Hansen, Herlev (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsværd (DK)

(21) Appl. No.: 11/908,835

(22) PCT Filed: Mar. 20, 2006
(86) PCT No.: PCT/EP2006/060854

§ 371 Date: Nov. 19, 2007

Related U.S. Application Data
(60) Provisional application No. 60/664,496, filed on Mar. 23, 2005.
(60) Provisional application No. 60/666,751, filed on Mar. 30, 2005.

(30) Foreign Application Priority Data
Mar. 18, 2005 (EP) 05102170.7
Mar. 30, 2005 (EP) 05102500.5

(51) Int. Cl. C07K 14/605; A61K 38/26; A61P 3/00; A61P 3/10; A61P 1/00; A61P 9/00

(52) U.S. Cl. 514/12; 530/323

(57) ABSTRACT

Dimerization of GLP-1 agonists and therapeutic uses thereof.

### FIELD OF THE INVENTION

This invention relates to the field of therapeutic peptides, i.e. to new GLP-1 agonists.

### BACKGROUND OF THE INVENTION

Diabetes mellitus is a metabolic disorder in which the ability to utilize glucose is partly or completely lost. About 5% of all people suffer from diabetes and the disorder approaches epidemic proportions. Since the introduction of insulin in the 1920's, continuous efforts have been made to improve the treatment of diabetes mellitus.

One peptide expected to become very important in the treatment of diabetes is glucagon-like peptide-1 (GLP-1). Human GLP-1 is a 37 amino acid residue peptide originating from preproglucagon which is synthesized i.a. in the L-cells in the distal ileum, in the pancreas and in the brain. GLP-1 is an important gut hormone with regulatory function in glucose metabolism and gastrointestinal secretion and metabolism. GLP-1 stimulates insulin secretion in a glucose-dependant manner, stimulates insulin biosynthesis, promotes beta cell rescue, decreases glucagon secretion, gastric emptying and food intake. Human GLP-1 is hydrolysed to GLP-1(7-37) and GLP-1(7-36)-amide which are both insulinotropic peptides. A simple system is used to describe fragments and analogues of this peptide. Thus, for example, [Gly⁸]GLP-1(7-37) designates an analogue of GLP-1(7-37) formally derived from GLP-1(7-37) by substituting the naturally occurring amino acid residue in position 8 (Ala) by Gly. Similarly, (N^{ε34}-tetradecanoyl)[Lys³⁴]GLP-1(7-37) designates GLP-1(7-37) wherein the ε-amino group of the Lys residue in position 34 has been tetradecanoylated. PCT publications WO 98/08871 and WO 99/43706 disclose stable derivatives of GLP-1 analogues, which have a lipophilic substituent. These stable derivatives of GLP-1 analogues have a protracted profile of action compared to the corresponding GLP-1 analogues.

Since 1992 a number of peptides have been isolated from the venom of the Gila monster lizards (Heloderma suspectum and Heloderma horridum). Exendin-4 is a 39 amino acid residue peptide isolated from the venom of Heloderma suspectum, and this peptide shares 52% homology with GLP-1(7-37) in the overlapping region. Exendin-4 is a potent GLP-1 receptor agonist which has been shown to stimulate insulin release and ensuing lowering of the blood glucose level when injected into dogs. The group of exendin-4(1-39), certain fragments thereof, analogs thereof and derivatives thereof, are potent insulinotropic agents. Most importantly the group of exendin-4(1-39), insulinotropic fragments thereof, insulinotropic analogs thereof and insulinotropic derivatives thereof.

The insulinotropic peptides derived from GLP-1 and Exendin-4 stimulate insulin release only when plasma glucose levels are high, the risk of hypoglycaemic events is reduced. Thus, the peptides are particularly useful for patients with diabetes who no longer respond to OHA's (oral hyperglycaemic agents) and who should from a strict medical point of view be administered insulin. Patients and to some extent also doctors are often not keen on initiating insulin treatment before this is absolutely necessary, presumably because of the fear of hypoglycaemic events or the fear of injections/needles. Thus, there is a need for insulinotropic peptides which are sufficiently potent and which can be administered by the pulmonary route.

Pulmonary administration of GLP-1 peptides have been disclosed in WO 01/51071 and WO 00/12116.

Thus, it is an object of the present invention to provide insulinotropic peptides which have sufficient pulmonary bioavailability to serve as an alternative to peptides for paranteral administration. Insulinotropic peptides having pulmonary bioavailability is a balance between potency and bioavailability. It is also an object of the present invention to provide insulinotropic peptides which are less prone to aggregation, a well known problem associated with the glucagon-like peptides. Being less prone to aggregation facilitates economical manufacturing processes as well as enabling the compounds to be administered by medical infusion pumps.

It is a further object of the invention to provide insulinotropic agent which have prolonged plasma half-life and which can thus be administered less than once daily.

### SUMMARY OF THE INVENTION

The present invention provides a compound which comprises two GLP-1 agonists linked to each other via a bifunctional cross-linker.

In one embodiment the two GLP-1 agonists are identical.

In another embodiment the two GLP-1 agonists are linked to the bifunctional crosslinker on the same amino acid residue.

In another embodiment said GLP-1 agonists are GLP-1 or an analogue thereof.

In another embodiment said GLP-1 agonists are exendin-4 or an analogue thereof.

In another aspect the invention provides a method for increasing the pulmonal bioavailability in a patient of a GLP-1 agonist, characterised in dimerisation of said GLP-1 agonist via a bifunctional crosslinker so as to produce a compound according to the present invention.

In another aspect the invention provides a method for increasing the ratio of pulmonal bioavailability to potency in a patient of a GLP-1 agonist, characterised in dimerisation of said GLP-1 agonist via a bifunctional crosslinker so as to produce a compound according to the present invention.

The present invention also provides pharmaceutical compositions comprising a compound according to the present invention and the use of compounds according to the present invention for preparing medicaments for treating disease.

### DEFINITIONS

In the present specification, the following terms have the indicated meaning:

The term “polypeptide” and “peptide” as used herein means a compound composed of at least five constituent amino acids connected by peptide bonds. The constituent amino acids may be from the group of the amino acids encoded by the genetic code and they may natural amino acids which are not encoded by the genetic code, as well as synthetic amino acids. Natural amino acids which are not encoded by the genetic code are e.g. hydroxyproline, γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), β-alanine, 3-aminomethyl benzoic acid, anthranilic acid.

The term “analogue” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. A simple system is often used to describe analogues: For example [Arg³⁴]GLP-1(7-37)Lys designates a GLP-1(7-37) analogue wherein the naturally occurring lysine at position 34 has been substituted with arginine and wherein a lysine has been added to the terminal amino acid residue, i.e. to the Gly³⁷. All amino acids for which the optical isomer is not stated is to be understood to mean the L-isomer. The term “derivative” as used herein in relation to a peptide means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters and the like. An example of a derivative of GLP-1(7-37) is N^{ε26}-((4S)-4-(hexadecanoylamino)-butanoyl)[Arg³⁴, Lys²⁶]GLP-1-(7-37).

The term “insulinotropic agent” as used herein means a compound which is an agonist of the human GLP-1 receptor, i.e. a compound which stimulates the formation of cAMP in a suitable medium containing the human GLP-1 receptor (one such medium disclosed below). The potency of an insulinotropic agent is determined by calculating the EC₅₀ value from the dose-response curve as described below.

Baby hamster kidney (BHK) cells expressing the cloned human GLP-1 receptor (BHK-467-12A) were grown in DMEM media with the addition of 100 IU/mL penicillin, 100 μg/mL streptomycin, 5% fetal calf serum and 0.5 mg/mL Geneticin G-418 (Life Technologies). The cells were washed twice in phosphate buffered saline and harvested with Versene. Plasma membranes were prepared from the cells by homogenisation with an Ultraturrax in buffer 1 (20 mM HEPES-Na, 10 mM EDTA, pH 7.4). The homogenate was centrifuged at 48,000×g for 15 min at 4° C. The pellet was suspended by homogenization in buffer 2 (20 mM HEPES-Na, 0.1 mM EDTA, pH 7.4), then centrifuged at 48,000×g for 15 min at 4° C. The washing procedure was repeated one more time. The final pellet was suspended in buffer 2 and used immediately for assays or stored at −80° C.

The functional receptor assay was carried out by measuring cyclic AMP (cAMP) as a response to stimulation by the insulinotropic agent. cAMP formed was quantified by the AlphaScreen™ cAMP Kit (Perkin Elmer Life Sciences). Incubations were carried out in half-area 96-well microtiter plates in a total volume of 50 μL buffer 3 (50 mM Tris-HCl, 5 mM HEPES, 10 mM MgCl₂, pH 7.4) and with the following addiditions: 1 mM ATP, 1 μM GTP, 0.5 mM 3-isobutyl-1-methylxanthine (IBMX), 0.01% Tween-20, 0.1% BSA, 6 μg membrane preparation, 15 μg/mL acceptor beads, 20 μg/mL donor beads preincubated with 6 nM biotinyl-cAMP. Compounds to be tested for agonist activity were dissolved and diluted in buffer 3. GTP was freshly prepared for each experiment. The plate was incubated in the dark with slow agitation for three hours at room temperature followed by counting in the Fusion™ instrument (Perkin Elmer Life Sciences). Concentration-response curves were plotted for the individual compounds and EC₅₀ values estimated using a four-parameter logistic model with Prism v. 4.0 (GraphPad, Carlsbad, Calif.).

The term “GLP-1 peptide” as used herein means GLP-1(7-37) (SEQ ID No 1), a GLP-1(7-37) analogue, a GLP-1(7-37) derivative or a derivative of a GLP-1(7-37) analogue. In one embodiment the GLP-1 peptide is an insulinotropic agent.

The term “exendin-4 peptide” as used herein means exendin-4(1-39) (SEQ ID No 2), an exendin-4(1-39) analogue, an exendin-4(1-39) derivative or a derivative of an exendin-4(1-39) analogue. In one embodiment the exendin-4 peptide is an insulinotropic agent.

The term “DPP-IV protected” as used herein referring to a polypeptide means a polypeptide which has been chemically modified in order to render said compound resistant to the plasma peptidase dipeptidyl aminopeptidase-4 (DPP-IV). The DPP-IV enzyme in plasma is known to be involved in the degradation of several peptide hormones, e.g. GLP-1, GLP-2, Exendin-4 etc. Thus, a considerable effort is being made to develop analogues and derivatives of the polypeptides susceptible to DPP-IV mediated hydrolysis in order to reduce the rate of degradation by DPP-IV. In one embodiment a DPP-IV protected peptide is more resistant to DPP-IV than GLP-1(7-37) or Exendin-4(1-39).

Resistance of a peptide to degradation by dipeptidyl aminopeptidase IV is determined by the following degradation assay:

Aliquots of the peptide (5 nmol) are incubated at 37° C. with 1 μL of purified dipeptidyl aminopeptidase IV corresponding to an enzymatic activity of 5 mU for 10-180 minutes in 100 μL of 0.1 M triethylamine-HCl buffer, pH 7.4. Enzymatic reactions are terminated by the addition of 5 μL of 10% trifluoroacetic acid, and the peptide degradation products are separated and quantified using HPLC analysis. One method for performing this analysis is The mixtures are applied onto a Vydac C18 widepore (30 nm pores, 5 μm particles) 250×4.6 mm column and eluted at a flow rate of 1 ml/min with linear stepwise gradients of acetonitrile in 0.1% trifluoroacetic acid (0% acetonitrile for 3 min, 0-24% acetonitrile for 17 min, 24-48% acetonitrile for 1 min) according to Siegel et al., Regul. Pept. 1999; 79:93-102 and Mentlein et al. Eur. J. Biochem. 1993; 214:829-35. Peptides and their degradation products may be monitored by their absorbance at 220 nm (peptide bonds) or 280 nm (aromatic amino acids), and are quantified by integration of their peak areas related to those of standards. The rate of hydrolysis of a peptide by dipeptidyl aminopeptidase IV is estimated at incubation times which result in less than 10% of the peptide being hydrolysed.

The term “C₁₋₆-alkyl” as used herein means a saturated, branched, straight or cyclic hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl, cyclohexane and the like.

The term “pharmaceutically acceptable” as used herein means suited for normal pharmaceutical applications, i.e. giving rise to no adverse events in patients etc.

The term “excipient” as used herein means the chemical compounds which are normally added to pharmaceutical compositions, e.g. buffers, tonicity agents, preservatives and the like.

The term “effective amount” as used herein means a dosage which is sufficient to be effective for the treatment of the patient compared with no treatment.

The term “pharmaceutical composition” as used herein means a product comprising an active compound or a salt thereof together with pharmaceutical excipients such as buffer, preservative, and optionally a tonicity modifier and/or a stabilizer. Thus a pharmaceutical composition is also known in the art as a pharmaceutical formulation.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

### DESCRIPTION OF THE INVENTION

In one aspect the present invention relates to a compound which comprises two GLP-1 agonists linked to each other via a bifunctional cross-linker.

In one embodiment the two GLP-1 agonists are identical.

In another embodiment the two GLP-1 agonists are linked to the bifunctional crosslinker on the same amino acid residue.

In another embodiment said GLP-1 agonists are GLP-1 or an analogue thereof.

In another embodiment said GLP-1 agonists are exendin-4 or an analogue thereof.

In another embodiment the two GLP-1 agonists are linked via a bifunctional hydrophilic spacer W—(CH₂)ₗD[(CH₂)ₙE]ₘ(CH₂)ₚQ_{q}-, wherein

l, m and n independently are 1-20 and p is 0-10,
Q is -Z-(CH₂)ₗD[(CH₂)ₙG]ₘ(CH₂)ₚ—,
W is —(CH₂)ₚ[(CH₂)ₙG]ₘD(CH₂)ₗ-Z-,
q is an integer in the range from 0 to 5,
each D, E, and G independently are selected from —O—, —NR³—, —N(COR⁴)—, —PR⁵(O)—, and —P(OR⁶)(O)—, wherein R³, R⁴, R⁵, and R⁶ independently represent hydrogen or C₁₋₆-alkyl,
Z is selected from —C(O)NH—, —C(O)NHCH₂—, —OC(O)NH—, —C(O)NHCH₂CH₂—, —C(O)CH₂—, —C(O)CH═CH—, —(CH₂)ₛ—, —C(O)—, —C(O)O— or —NHC(O)—, wherein s is 0 or 1.

In a further aspect the invention relates to a compound of the formula (I):

GLP-1 compound-Y—B—Y-GLP-1 compound* (I)

wherein
B is a hydrophilic spacer being W_{q}—(CH₂)ₗD[(CH₂)ₙE]ₘ(CH₂)ₚQ_{q}-,
l, m and n independently are 1-20 and p is 0-10,
Q is -Z-(CH₂)ₗD[(CH₂)ₙG]ₘ(CH₂)ₚ—,
W is —(CH₂)ₚ[(CH₂)ₙG]ₘD(CH₂)ₗ-Z-,
q is an integer in the range from 0 to 5,
each D, E, and G independently are selected from —O—, —NR³—, —N(COR⁴)—, —PR⁵(O)—, and —P(OR⁶)(O)—, wherein R³, R⁴, R⁵, and R⁶ independently represent hydrogen or C₁₋₆-alkyl,
Z is selected from —C(O)NH—, —C(O)NHCH₂—, —OC(O)NH—, —C(O)NHCH₂CH₂—, —C(O)CH₂—, —C(O)CH═CH—, —(CH₂)ₛ—, —C(O)—, —C(O)O— or —NHC(O)—, wherein s is 0 or 1,
Y is a chemical group linking B and the GLP-1 agonists,
“GLP-1 compound” and “GLP-1 compound*” are GLP-1 agonists.

In a further aspect the present invention relates to a compound which has the formula (II)

GLP-1 compound-Y—B—B′—Y′-GLP-1 compound* (II)

wherein
B and B′ are hydrophilic spacers independently selected from —W_{q}—(CH₂)ₗD[(CH₂)ₙE]ₘ(CH₂)ₚ-Q_{q}-, wherein
l, m and n independently are 1-20 and p is 0-10,
Q is -Z-(CH₂)ₗD[(CH₂)ₙG]ₘ(CH₂)ₚ—,
W is —(CH₂)ₚ[(CH₂)ₙG]ₘD(CH₂)ₗ-Z-
q is an integer in the range from 0 to 5,
each D, E, and G independently are selected from —O—, —NR³—, —N(COR⁴)—, —PR⁵(O)—, and —P(OR⁶)(O)—, wherein R³, R⁴, R⁵, and R⁶ independently represent hydrogen or C₁₋₆-alkyl,
Z is selected from —C(O)NH—, —C(O)NHCH₂—, —OC(O)NH—, —C(O)NHCH₂CH₂—, —C(O)CH₂—, —C(O)CH═CH—, —(CH₂)ₛ—, —C(O)—, —C(O)O— or —NHC(O)—, wherein s is 0 or 1,
Y is a chemical group linking B and the GLP-1 agonist, and
Y′ is a chemical group linking B′ and the GLP-1 agonist, and
“GLP-1 compound” and “GLP-1 compound*” are GLP-1 agonists.

In the embodiments above the two GLP-1 compounds may be identical or different.

In another embodiment Y′ and Y′ are selected from the group consisting of —C(O)NH—, —NHC(O)—, —C(O)NHCH₂—, —CH₂NHC(O)—, —OC(O)NH—, —NHC(O)O—, —C(O)NHCH₂—, CH₂NHC(O)—, —C(O)CH₂—, —CH₂C(O)—, —C(O)CH═CH—, —CH═CHC(O)—, —(CH₂)ₛ—, —C(O)—, —C(O)O—, —OC(O)—, —NHC(O)— and —C(O)NH—, wherein s is 0 or 1.

In another embodiment I is 1 or 2, n and m are independently 1-10 and p is 0-10.

In another embodiment D is —O—.

In another embodiment E is —O—.

In another embodiment the hydrophilic spacer is —CH₂O[(CH₂)₂O]ₘ(CH₂)ₚQ_{q}-, where m is 1-10, p is 1-3, and Q is -Z-CH₂O[(CH₂)₂O]ₘ(CH₂)ₚ—.

In another embodiment q is 0.

In another embodiment q is 1.

In another embodiment G is —O—.

In another embodiment Z is selected from the group consisting of —C(O)NH—, —C(O)NHCH₂—, and —OC(O)NH—.

In another embodiment I is 2.

In another embodiment n is 2.

In another embodiment the hydrophilic spacer B is —[CH₂CH₂O]ₘ₊₁(CH₂)ₚQ_{q}-.

In an embodiment the compound according to any of the preceding embodiments is a GLP-1 compound comprises the amino acid sequence of formula I:

Formula (I)

(SEQ. ID No: 1)

Xaa₁-Xaa₂-His-Gly-Xaa₅-Phe-Xaa₇-Xaa₈-Xaa₉-Xaa₁₀-
Xaa₁₁-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Phe-Xaa₂₃-Xaa₂₄-Trp-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉-Xaa₄₀-Xaa₄₁-Xaa₄₂

wherein
Xaa₁ is L-histidine, D-histidine, desamino-histidine, 2-amino-3-(2-aminoimidazol-4-yl)propionic acid, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine; or L-tyrosine
Xaa₂ is Ala, Gly, Val, Leu, Ile, Lys, Aib, 1-aminocyclopropanecarboxylic acid, 1-aminocyclobutanecarboxylic acid, 1-aminocyclopentanecarboxylic acid, 1-aminocyclohexanecarboxylic acid, 1-aminocycloheptanecarboxylic acid, or 1-aminocyclooctanecarboxylic acid;

### Xaa₅ is Thr or Ser;

### Xaa₇ is Thr or Ser;

### Xaa₈ is Ser or Asp;

### Xaa₉ is Glu or Asp;

### Xaa₁₀ is Val, Met, Leu or Tyr;

### Xaa₁₁ is Ser, or Asn;

### Xaa₁₂ is Ser, Thr, Lys or Ile;

### Xaa₁₃ is Tyr, Ile, Ala or Gln;

### Xaa₁₄ is Leu or Met;

### Xaa₁₅ is Asp or Glu;

### Xaa₁₆ is Gly, Asn, Glu or Lys;

### Xaa₁₇ is Leu, Gln, Glu or Ile;

### Xaa₁₈ is Ala or His;

### Xaa₁₉ is Ala, Gln or Val;

### Xaa₂₀ is Lys, Arg or Gln;

### Xaa₂₁ is Asp, Glu or Leu;

### Xaa₂₃ is Ile or Val;

### Xaa₂₄ is Ala, Asn or Glu;

### Xaa₂₆ is Leu or Ile;

### Xaa₂₇ is Val, Ile, Leu, Arg or Lys;

### Xaa₂₈ is Lys, Gln, Ala or Asn;

### Xaa₂₉ is Gly, Thr or Gln;

### Xaa₃₀ is Arg, Lys or Gly;

Xaa₃₁ is Ile, Gly, Pro, amide or is absent;
Xaa₃₂ is Thr, Lys, Ser, amide or is absent;
Xaa₃₃ is Asp, Lys, Ser, amide or is absent;
Xaa₃₄ is Arg, Asn, Gly, amide or is absent;
Xaa₃₅ is Asp, Ala, amide or is absent;
Xaa₃₆ is Trp, Pro, amide or is absent;
Xaa₃₇ is Lys, Pro, amide or is absent;
Xaa₃₈ is His, Pro, amide or is absent;
Xaa₃₉ is Asn, Ser, amide or is absent;
Xaa₄₀ is Ile, amide or is absent;
Xaa₄₁ is Thr, amide or is absent;
Xaa₄₂ is Gln, amide or is absent;
provided that if Xaa₃₁, Xaa₃₂, Xaa₃₃, Xaa₃₄, Xaa₃₅, Xaa₃₆, Xaa₃₇, Xaa₃₈, Xaa₃₉, Xaa₄₀, Xaa₄₁, or Xaa₄₂ is absent then each amino acid residue downstream is also absent.

In an embodiment the amino acid sequence is according to formula 2:

Formula (2)

(SEQ. ID No: 2)

His-Xaa₂-His-Gly-Xaa₅-Phe-Xaa₇-Xaa₈-Xaa₉-Xaa₁₀-
Xaa₁₁-Xaa₁₂-Xaa₁₃-Xaa₁₄-Xaa₁₅-Xaa₁₆-Xaa₁₇-Ala-
Xaa₁₉-Xaa₂₀-Xaa₂₁-Phe-Ile-Xaa₂₄-Trp-Leu-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉

wherein
Xaa₂ is Ala, Gly, Val, Leu, Ile, Lys, Aib, 1-aminocyclopropanecarboxylic acid, 1-aminocyclobutanecarboxylic acid, 1-aminocyclopentanecarboxylic acid, 1-aminocyclohexanecarboxylic acid, 1-aminocycloheptanecarboxylic acid, or 1-aminocyclooctanecarboxylic acid;

### Xaa₅ is Thr or Ser;

### Xaa₇ is Thr or Ser;

### Xaa₈ is Ser or Asp;

### Xaa₉ is Glu or Asp;

### Xaa₁₀ is Val, Met, or Leu;

### Xaa₁₁ is Ser or Asn;

### Xaa₁₂ is Ser, Thr or Lys;

### Xaa₁₃ is Tyr, Ile or Gln;

### Xaa₁₄ is Leu or Met;

### Xaa₁₅ is Asp or Glu;

### Xaa₁₆ is Gly, Asn or Glu;

### Xaa₁₇ is Leu, Gln or Glu;

### Xaa₁₉ is Ala or Val;

### Xaa₂₀ is Lys or Arg;

### Xaa₂₁ is Asp, Glu or Leu;

### Xaa₂₄ is Ala, Asn or Glu;

### Xaa₂₇ is Val, Ile or Lys;

### Xaa₂₈ is Lys, Gln or Asn;

### Xaa₂₉ is Gly or Thr;

### Xaa₃₀ is Arg, Lys or Gly;

Xaa₃₁ is Ile, Pro, amide or is absent;
Xaa₃₂ is Thr, Ser, amide or is absent;
Xaa₃₃ is Asp, Ser, amide or is absent;
Xaa₃₄ is Arg, Gly, amide or is absent;
Xaa₃₅ is Ala, amide or is absent;
Xaa₃₆ is Pro, amide or is absent;
Xaa₃₇ is Pro, amide or is absent;
Xaa₃₈ is Pro, amide or is absent;
Xaa₃₉ is Ser, amide or is absent;
provided that if Xaa₃₁, Xaa₃₂, Xaa₃₃, Xaa₃₄, Xaa₃₅, Xaa₃₆, Xaa₃₇, Xaa₃₈, or Xaa₃₉ is absent then each amino acid residue downstream is also absent.

In an embodiment the amino acid sequence is according to formula 3:

Formula (3)

(SEQ. ID No: 3)

His-Xaa₂-His-Gly-Thr-Phe-Thr-Ser-Asp-Xaa₁₀-Ser-
Xaa₁₂-Xaa₁₃-Xaa₁₄-Glu-Xaa₁₆-Xaa₁₇-Ala-Xaa₁₉-Xaa₂₀-
Xaa₂₁-Phe-Ile-Xaa₂₄-Trp-Leu-Xaa₂₇-Xaa₂₈-Gly-Xaa₃₀-
Xaa₃₁-Xaa₃₂-Xaa₃₃-Xaa₃₄-Xaa₃₅-Xaa₃₆-Xaa₃₇-Xaa₃₈-
Xaa₃₉

Xaa₂ is Ala, Gly, Val, Leu, Ile, Lys, Aib, 1-aminocyclopropanecarboxylic acid, 1-aminocyclobutanecarboxylic acid, 1-aminocyclopentanecarboxylic acid, 1-aminocyclohexanecarboxylic acid, 1-aminocycloheptanecarboxylic acid, or 1-aminocyclooctanecarboxylic acid;

### Xaa₁₀ is Val or Leu;

### Xaa₁₂ is Ser or Lys;

### Xaa₁₃ is Tyr or Gln;

### Xaa₁₄ is Leu or Met;

### Xaa₁₆ is Gly or Glu;

### Xaa₁₇ is Gln or Glu;

### Xaa₁₉ is Ala or Val;

### Xaa₂₀ is Lys or Arg;

### Xaa₂₁ is Glu or Leu;

### Xaa₂₄ is Ala or Glu;

### Xaa₂₇ is Val or Lys;

### Xaa₂₈ is Lys or Asn;

### Xaa₃₀ is Arg, Lys or Gly;

Xaa₃₁ is Pro, amide or is absent;
Xaa₃₂ is Ser, amide or is absent;
Xaa₃₃ is Ser, amide or is absent;
Xaa₃₄ is Gly, amide or is absent;
Xaa₃₅ is Ala, amide or is absent;
Xaa₃₆ is Pro, amide or is absent;
Xaa₃₇ is Pro, amide or is absent;
Xaa₃₈ is Pro, amide or is absent;
Xaa₃₉ is Ser, amide or is absent;
provided that if Xaa₃₁, Xaa₃₂, Xaa₃₃, Xaa₃₄, Xaa₃₅, Xaa₃₆, Xaa₃₇, Xaa₃₈, or Xaa₃₉ is absent then each amino acid residue downstream is also absent.

In another embodiment the two GLP-1 agonists are dimerised via amino acid residue at one of the following positions:

GLP-1: residue number 18, 22, 26, 34, 36, 37 or 38
Exendin-4: residue number 12, 16, 20, 32, 33 or 34.

In another embodiment the invention relates to a compound according to any one of the previous claims which is selected from
N,N′-Bis-[epsilon,26-desamino Arg34,Lys26-GLP-1(7-37)-epsilon,26-yl]-O,O′-1,13-dideoxy-tetraethylenglycol-hydracrylic amide
N,N′-Bis-[epsilon,26-desamino Arg34,Lys26-GLP-1(7-37)-epsilon,26-yl]-O,O′-1,14-dideoxy-tetraethylenglycol-hydracrylic amide

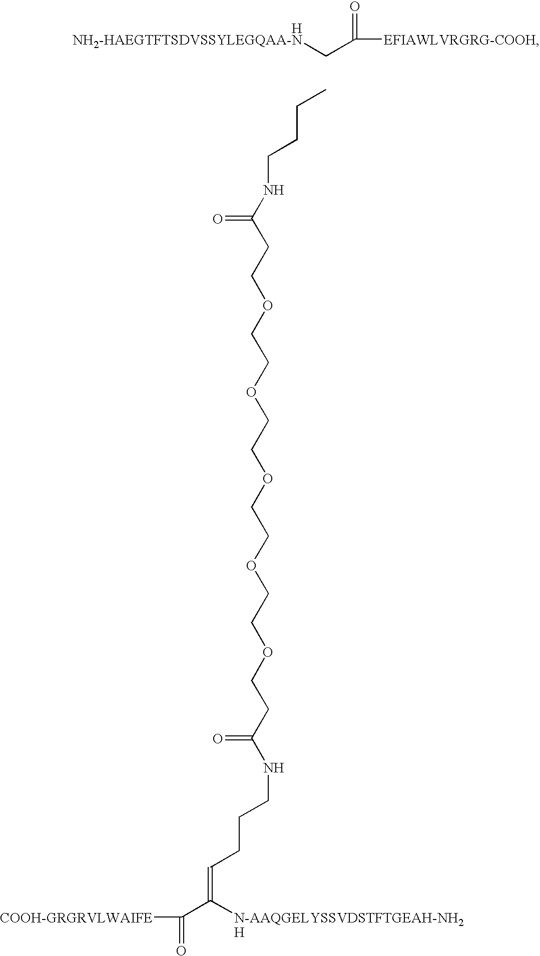

N,N′-Bis-[epsilon,34-desamino {Arg12, Leu14, Arg27, Lys34} Exendin-4 (1-39)-amide-epsilon,34-yl]-O,O′-1,13-dideoxy-tetraethylenglycol-hydracrylic amide
N,N′-Bis-[epsilon,26-desamino Arg34,Lys26-GLP-1(7-37)-epsilon,26-yl]-O,O′-1,14-dideoxy-tetraethylenglycol-hydracrylic amide

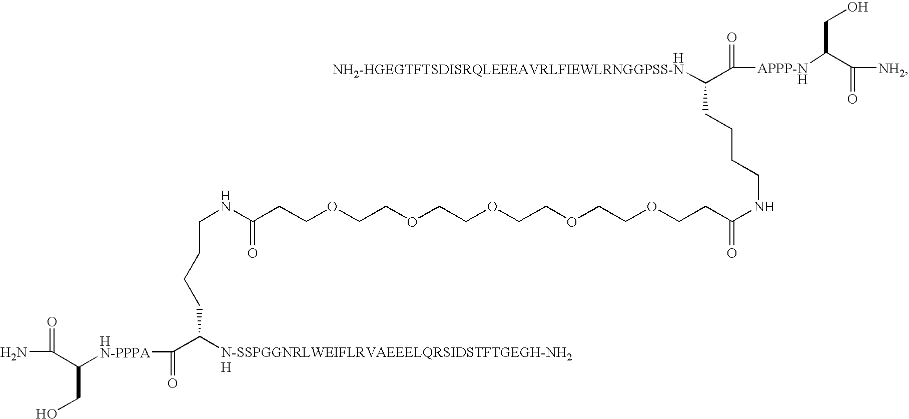

N,N′-Bis-[epsilon,20-desamino Arg26, Arg34, Lys20-GLP-1(7-37)-epsilon,20-yl]-O,O′-1,13-dideoxy-tetraethylenglycol-hydracrylic amide,
N,N′-Bis-[epsilon,38-desamino Arg26, Arg34, Lys38-GLP-1(7-37)-epsilon,38-yl]-O,O′-1,13-dideoxy-tetraethylenglycol-hydracrylic amide,
N,N′-Bis-[epsilon,18-desamino Arg26, Arg34, Lys16-GLP-1(7-37)-epsilon,18-yl]-O,O′-1,13-dideoxy-tetraethylenglycol-hydracrylic amide,
N,N′-Bis-[epsilon,39-desamino {Arg12, Leu14, Arg27, Lys39} Exendin-4 (1-39)-amide-epsilon,39-yl]-O,O′-1,13-dideoxy-tetraethylenglycol-hydracrylic amide, and
N,N′-Bis-[epsilon,20-desamino {Arg12, Leu14, Arg27, Lys20} Exendin-4 (1-39)-amide-epsilon,20-yl]-O,O′-1,13-dideoxy-tetraethylenglycol-hydracrylic amide.
N,N′-Bis-[epsilon,26-desamino desamino-His7,Arg34,Lys26-GLP-1(7-37)-epsilon,26-yl]-O,O′-1,13-dideoxy-tetraethylenglycol-hydracrylic amide
N,N′-Bis-[epsilon,20-desamino desamino-His7,Arg26, Arg34, Lys20-GLP-1(7-37)-epsilon,20-yl]-O,O′-1,13-dideoxy-tetraethylenglycol-hydracrylic amide,
N,N′-Bis-[epsilon,38-desamino desamino-His7,Arg26, Arg34, Lys38-GLP-1(7-37)-epsilon,38-yl]-O,O′-1,13-dideoxy-tetraethylenglycol-hydracrylic amide,
N,N′-Bis-[epsilon,18-desamino desamino-His7,Arg26, Arg34, Lys16-GLP-1(7-37)-epsilon,18-yl]-O,O′-1,13-dideoxy-tetraethylenglycol-hydracrylic amide,
N,N′-Bis-[epsilon,26-desamino Aib8,Arg34,Lys26-GLP-1(7-37)-epsilon,26-yl]-O,O′-1,13-dideoxy-tetraethylenglycol-hydracrylic amide

N,N′-Bis-[epsilon,20-desamino Aib8,Arg26, Arg34, Lys20-GLP-1(7-37)-epsilon,20-yl]-O,O′-1,13-dideoxy-tetraethylenglycol-hydracrylic amide,
N,N′-Bis-[epsilon,38-desamino Aib8,Arg26, Arg34, Lys38-GLP-1(7-37)-epsilon,38-yl]-O,O′-1,13-dideoxy-tetraethylenglycol-hydracrylic amide,
N,N′-Bis-[epsilon,18-desamino Aib8,Arg26, Arg34, Lys16-GLP-1(7-37)-epsilon,18-yl]-O,O′-1,13-dideoxy-tetraethylenglycol-hydracrylic amide,
N,N′-Bis-[epsilon,26-desamino Gly8,Arg34,Lys26-GLP-1(7-37)-epsilon,26-yl]-O,O′-1,13-dideoxy-tetraethylenglycol-hydracrylic amide
N,N′-Bis-[epsilon,20-desamino Gly8,Arg26, Arg34, Lys20-GLP-1(7-37)-epsilon,20-yl]-O,O′-1,13-dideoxy-tetraethylenglycol-hydracrylic amide,
N,N′-Bis-[epsilon,38-desamino Gly8,Arg26, Arg34, Lys38-GLP-1(7-37)-epsilon,38-yl]-O,O′-1,13-dideoxy-tetraethylenglycol-hydracrylic amide,
N,N′-Bis-[epsilon,18-desamino Gly8,Arg26, Arg34, Lys16-GLP-1(7-37)-epsilon,18-yl]-O,O′-1,13-dideoxy-tetraethylenglycol-hydracrylic amide,
N,N′-Bis-[epsilon,26-desamino Val8,Arg34,Lys26-GLP-1(7-37)-epsilon,26-yl]-O,O′-1,13-dideoxy-tetraethylenglycol-hydracrylic amide
N,N′-Bis-[epsilon,20-desamino Val8,Arg26, Arg34, Lys20-GLP-1(7-37)-epsilon,20-yl]-O,O′-1,13-dideoxy-tetraethylenglycol-hydracrylic amide,
N,N′-Bis-[epsilon,38-desamino Val8,Arg26, Arg34, Lys38-GLP-1(7-37)-epsilon,38-yl]-O,O′-1,13-dideoxy-tetraethylenglycol-hydracrylic amide,
N,N′-Bis-[epsilon,18-desamino Val8,Arg26, Arg34, Lys16-GLP-1(7-37)-epsilon,18-yl]-O,O′-1,13-dideoxy-tetraethylenglycol-hydracrylic amide,
N,N′-bis((((S)-5-([Aib,8,22,35]GLP-1(1-37)yl)-5-carbamoylpentyl) carbamoyl)methoxy)hexan-1,6-diimine
N,N′-bis((((S)-5-(N-epsilon26[2-(2-[2-(2-[2-(2[4-(17-carboxyheptadecanoylamino)-4(S)-carboxybutyrylamino]ethoxy)ethoxy]acetylamino)ethoxy]ethoxy)acetyl][Aib8,Arg34]GLP-1(7-37) yl)-5-carbamoylpentyl)carbamoyl)methoxy)ethan-1,2-diimine

In another aspect the invention relates to a method for increasing the pulmonal bioavailability in a patient of a GLP-1 agonist, characterised in dimerisation of said GLP-1 agonist via a bifunctional crosslinker so as to produce a compound according to the invention.

In another aspect the invention relates to a method for increasing the ratio of pulmonal bioavailability to potency in a patient of a GLP-1 agonist, characterised in dimerisation of said GLP-1 agonist via a bifunctional crosslinker so as to produce a compound according to the invention.

Another object of the present invention is to provide a pharmaceutical formulation comprising a compound according to the present invention which is present in a concentration from 0.1 mg/ml to 25 mg/ml, and wherein said formulation has a pH from 3.0 to 9.0. The formulation may further comprise a buffer system, preservative(s), tonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further aspect the invention relates to a pharmaceutical formulation comprising an aqueous solution of a compound according to the present invention, and a buffer, wherein said compound is present in a concentration from 0.1 mg/ml or above, and wherein said formulation has a pH from about 3.0 to about 9.0.

In another embodiment of the invention the pH of the formulation is from about 7.0 to about 9.5. In another embodiment of the invention the pH of the formulation is from about 3.0 to about 7.0. In another embodiment of the invention the pH of the formulation is from about 5.0 to about 7.5. In another embodiment of the invention the pH of the formulation is from about 7.5 to about 9.0. In another embodiment of the invention the pH of the formulation is from about 7.5 to about 8.5. In another embodiment of the invention the pH of the formulation is from about 6.0 to about 7.5. In another embodiment of the invention the pH of the formulation is from about 6.0 to about 7.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one—OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

In a further embodiment of the invention the formulation further comprises a stabilizer. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or a mixture thereof) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L or D) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)—derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

In a further embodiment of the invention the formulation further comprises protease inhibitors such as EDTA (ethylenediamine tetraacetic acid) and benzamidineHCl, but other commercially available protease inhibitors may also be used. The use of a protease inhibitor is particular useful in pharmaceutical compositions comprising zymogens of proteases in order to inhibit autocatalysis.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a compound according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the compound of the present invention, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of compounds of the present invention, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles, Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000). Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the compound of the present invention in the form of a nasal or pulmonal spray. As a still further option, the pharmaceutical compositions containing the compound of the invention can also be adapted to transdermal administration, e.g. by needle-free injection or from a patch, optionally an iontophoretic patch, or transmucosal, e.g. buccal, administration.

The compounds of the present invention can be administered via the pulmonary route in a vehicle, as a solution, suspension or dry powder using any of known types of devices suitable for pulmonary drug delivery. Examples of these comprise, but are not limited to, the three general types of aerosol-generating for pulmonary drug delivery, and may include jet or ultrasonic nebulizers, metered-dose inhalers, or dry powder inhalers (Cf. Yu J, Chien Y W. Pulmonary drug delivery: Physiologic and mechanistic aspects. Crit. Rev Ther Drug Carr Sys 14(4) (1997) 395-453).

Based on standardised testing methodology, the aerodynamic diameter (dₐ) of a particle is defined as the geometric equivalent diameter of a reference standard spherical particle of unit density (1 g/cm³). In the simplest case, for spherical particles, dₐ is related to a reference diameter (d) as a function of the square root of the density ratio as described by:

$${d}_{a} = \sqrt{\frac{ρ}{{ρ}_{a}}} d$$

Modifications to this relationship occur for non-spherical particles (cf. Edwards D A, Ben-Jebria A, Langer R. Recent advances in pulmonary drug delivery using large, porous inhaled particles. J Appl Physiol 84(2) (1998) 379-385). The terms “MMAD” and “MMEAD” are well-described and known to the art (cf. Edwards D A, Ben-Jebria A, Langer R and represents a measure of the median value of an aerodynamic particle size distribution. Recent advances in pulmonary drug delivery using large, porous inhaled particles. J Appl Physiol 84(2) (1998) 379-385). Mass median aerodynamic diameter (MMAD) and mass median effective aerodynamic diameter (MMEAD) are used inter-changeably, are statistical parameters, and empirically describe the size of aerosol particles in relation to their potential to deposit in the lungs, independent of actual shape, size, or density (cf. Edwards D A, Ben-Jebria A, Langer R. Recent advances in pulmonary drug delivery using large, porous inhaled particles. J Appl Physiol 84(2) (1998) 379-385). MMAD is normally calculated from the measurement made with impactors, an instrument that measures the particle inertial behaviour in air.

In a further embodiment, the formulation could be aerosolized by any known aerosolisation technology, such as nebulisation, to achieve a MMAD of aerosol particles less than 10 μm, more preferably between 1-5 μm, and most preferably between 1-3 μm. The preferred particle size is based on the most effective size for delivery of drug to the deep lung, where protein is optimally absorbed (cf. Edwards D A, Ben-Jebria A, Langer A, Recent advances in pulmonary drug delivery using large, porous inhaled particles. J Appl Physiol 84(2) (1998) 379-385).

Deep lung deposition of the pulmonal formulations comprising the compound of the present invention may optional be further optimized by using modifications of the inhalation techniques, for example, but not limited to: slow inhalation flow (eg. 30 L/min), breath holding and timing of actuation.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the compound of the present invention is stable for more than 6 weeks of usage and for more than 3 years of storage.

In another embodiment of the invention the pharmaceutical formulation comprising the compound of the present invention is stable for more than 4 weeks of usage and for more than 3 years of storage.

In a further embodiment of the invention the pharmaceutical formulation comprising the compound of the present invention is stable for more than 4 weeks of usage and for more than two years of storage.

In an even further embodiment of the invention the pharmaceutical formulation comprising the compound of the present invention is stable for more than 2 weeks of usage and for more than two years of storage.

In another aspect the present invention relates to the use of a compound according to the invention for the preparation of a medicament.

In one embodiment a compound according to the invention is used for the preparation of a medicament for the treatment or prevention of hyperglycemia, type 2 diabetes, impaired glucose tolerance, type 1 diabetes, obesity, hypertension, syndrome X, dyslipidemia, cognitive disorders, atheroschlerosis, myocardial infarction, stroke, coronary heart disease and other cardiovascular disorders, inflammatory bowel syndrome, dyspepsia and gastric ulcers.

In another embodiment a compound according to the invention is used for the preparation of a medicament for delaying or preventing disease progression in type 2 diabetes.

In another embodiment a compound according to the invention is used for the preparation of a medicament for decreasing food intake, decreasing β-cell apoptosis, increasing β-cell function and β-cell mass, and/or for restoring glucose sensitivity to β-cells.

The treatment with a compound according to the present invention may also be combined with a second or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: Insulin, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), Gastric Inhibitory Polypeptides (GIP analogs), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, PYY agonist, PYY2 agonists, PYY4 agonits, mixed PPY2/PYY4 agonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists, Gastric Inhibitory Polypeptide agonists or antagonists (GIP analogs), gastrin and gastrin analogs.

It should be understood that any suitable combination of the compounds according to the invention with one or more of the above-mentioned compounds and optionally one or more further pharmacologically active substances are considered to be within the scope of the present invention.

The present invention is further illustrated by the following examples which, however, are not to be construed as limiting the scope of protection. The features disclosed in the foregoing description and in the following examples may, both separately and in any combination thereof, be material for realising the invention in diverse forms thereof.

### EXAMPLES

The following acronyms for commercially available chemicals are used:

| | |
|---|---|
| DMF | N,N-Dimethylformamide. |
| DCC | N,N-Dicyclohexylcarbodiimide |
| NMP | N-Methyl-2-pyrrolidone. |
| TFA | Trifluoroacetic acid. |
| THF | Tetrahydrofuran |
| DIEA | diisopropylethylamine |
| H₂O | water |
| CH₃CN | acetonitrile |
| HBTU | 2-(1H-Benzotriazol-1-yl)-1,1,3,3 |
| | tetramethyluronium |
| | hexafluoro-phosphate |
| Fmoc | 9H-fluoren-9-ylmethoxycarbonyl |
| Boc | tert butyloxycarbonyl |
| OtBu | tert butyl ester |
| tBu | tert butyl |
| Trt | triphenylmethyl |
| Pmc | 2,2,5,7,8-Pentamethyl-chroman-6-sulfonyl |
| Dde | 1-(4,4-Dimethyl-2,6- |
| | dioxocyclohexylidene)ethyl |
| DCM | dichloromethane |
| TIS | triisopropylsilane) |
| Et₂O: | diethylether |
| H-Glu(OH)—OBu^{t}: | L-Glutamic acid α-tert-butyl ester |
| HOOC—(CH₂)₁₂—COONSu | ω-Carboxytridecanoic acid 2,5- |
| | dioxopyrrolidin-1-yl ester. |
| HOOC—(CH₂)₁₄—COONSu | ω\tilde{-}Carboxypentadecanoic acid 2,5- |
| | dioxopyrrolidin-1-yl ester. |
| HOOC—(CH₂)₁₆—COONSu | ω\tilde{-}Carboxyheptadecanoic acid 2,5- |
| | dioxopyrrolidin-1-yl ester. |
| HOOC—(CH₂)₁₈—COONSu | ω-Carboxynonadecanoic acid 2,5- |
| | dioxopyrrolidin-1-yl ester. |

### Abbreviations:

r.t Room temperature

### PDMS: Plasma Desorption Mass Spectrometry

### MALDI-MS: Matrix Assisted Laser Desorption/Ionisation Mass Spectrometry

### HPLC: High Performance Liquid Chromatography

amu: atomic mass units

### Analytical:

Resistance of a peptide to degradation by dipeptidyl aminopeptidase IV is determined by the following degradation assay:

Aliquots of the peptides are incubated at 37° C. with an aliquot of purified dipeptidyl aminopeptidase IV for 4-22 hours in an appropriate buffer at pH 7-8 (buffer not being albumin). Enzymatic reactions are terminated by the addition of trifluoroacetic acid, and the peptide degradation products are separated and quantified using HPLC or LC-MS analysis. One method for performing this analysis is: The mixtures are applied onto a Zorbax 300SB-C18 (30 nm pores, 5 μm particles) 150×2.1 mm column and eluted at a flow rate of 0.5 ml/min with a linear gradient of acetonitrile in 0.1% trifluoroacetic acid (0%-100% acetonitrile over 30 min). Peptides and their degradation products may be monitored by their absorbance at 214 nm (peptide bonds) or 280 nm (aromatic amino acids), and are quantified by integration of their peak areas. The degradation pattern can be determined by using LC-MS where MS spectra of the separated peak can be determined. Percentage intact/degraded compound at a given time is used for estimation of the peptides DPPIV stability.

A peptide is defined as DPPIV stabilised when it is 10 times more stable than the natural peptide based on percentage intact compound at a given time. Thus, a DPPIV stabilised GLP-1 compound is at least 10 times more stable than GLP-1(7-37).

### General Synthetic Methods

The peptides may be synthesized on Fmoc protected Rink amide resin (Novabiochem) or chlorotrityl resin or a similar resin suitable for solid phase peptide synthesis. Boc chemistry may be used but more conveinient is using Fmoc strategy eventually on an Applied Biosystems 433A peptide synthesizer in 0.25 mmol scale using the FastMoc UV protocols which employ HBTU (2-(1H-Benzotriazol-1-yl)-1,1,3,3 tetramethyluronium hexafluorophosphate) mediated couplings in N-methylpyrrolidone (N-methylpyrrolidone) (HATU is better suited for hindered couplings) and UV monitoring of the deprotection of the Fmoc protection group. Other coupling reagents besides from HBTU and HATU as described in e.g. Current Opinion in Chemical Biology, 2004, 8:211-221 may also be used. The protected amino acid derivatives used may be standard Fmoc-amino acids supplied in pre-weighed cartridges (Applied Biosystems) suitable for the ABI433A synthesizer with the exception of unnatural amino acids such as Fmoc-Aib-OH (Fmoc-aminoisobutyric acid) which are purchased from a supplier such as Bachem and transferred to empty cartridges. The last amino acid coupled may be Boc protected.

The attachment of side chains and linkers to specific lysine residues on the crude resin bound protected peptide may eventually be introduced in a specific position by incorporation of Fmoc-Lys(Dde)-OH during automated synthesis followed by selective deprotection with hydrazine. Other orthogonal protecting groups may be used on Lysine.

Procedure for removal of Dde-protection. The resin (0.25 mmol) may be placed in a manual shaker/filtration apparatus and treated with 2% hydrazine in N-methylpyrrolidone (20 ml, 2×12 min) to remove the DDE group and subsequently washed with N-methylpyrrolidone (4×20 ml).

### Procedure for Attachment of Sidechains to Lysine Residues.

The amino acid (4 molar equivalents relative to resin) may be dissolved in N-methyl pyrrolidone/methylene chloride (1:1, 10 ml). Hydroxybenzotriazole (HOBt) (4 molar equivalents relative to resin) and diisopropylcarbodiimide (4 molar equivalents relative to resin) is added and the solution was stirred for 15 min. The solution is added to the resin and diisopropyethylamine (4 molar equivalents relative to resin) is added. The resin is shaken 24 hours at room temperature. The resin is washed with N-methylpyrrolidone (2×20 ml), N-methylpyrrolidone/Methylene chloride (1:1) (2×20 ml) and methylene chloride (2×20 ml).

Procedure for removal of Fmoc-protection: The resin (0.25 mmol) is placed in a filter flask in a manual shaking apparatus and treated with N-methylpyrrolidone/methylene chloride (1:1) (2×20 ml) and with N-methylpyrrolidone (1×20 ml), a solution of 20% piperidine in N-methyl pyrrolidone (3×20 ml, 10 min each). The resin is washed with N-methylpyrrolidone (2×20 ml), N-methylpyrrolidone/methylene chloride (1:1) (2×20 ml) and methylene chloride (2×20 ml).

### Procedure for Cleaving the Peptide Off the Resin:

The peptide is cleaved from the resin by stirring for 180 min at room temperature with a mixture of trifluoroacetic acid, water and triisopropylsilane (95:2.5:2.5). The cleavage mixture is filtered and the filtrate is concentrated to an oil by a stream of nitrogen. The crude peptide is precipitated from this oil with 45 ml diethyl ether and washed 3 times with 45 ml diethyl ether.

Purification: The crude peptide may be purified by semi-preparative HPLC on a 20 mm×250 mm column packed with 7μ C-18 silica. Depending on the peptide one or two purification systems may used:
Ammonium sulphate: The column is equilibrated with 40% CH₃CN in 0.05M (NH₄)₂SO₄, which is adjusted to pH 2.5 with concentrated H₂SO₄. After drying the crude peptide is dissolved in 5 ml 50% acetic acid H₂O and diluted to 20 ml with H₂O and injected on the column which then is eluted with a gradient of 40%-60% CH₃CN in 0.05M (NH₄)₂SO₄, pH 2.5 at 10 ml/min during 50 min at 40° C. The peptide containing fractions is collected and diluted with 3 volumes of H₂O and passed through a Sep-Pak® C18 cartridge (Waters part. #:51910) which has been equilibrated with 0.1% TFA. It is then eluted with 70% CH₃CN containing 0.1% TFA and the purified peptide is isolated by lyophilisation after dilution of the eluate with water.
TFA: After drying the crude peptide is dissolved in 5 ml 50% acetic acid H₂O and diluted to 20 ml with H₂O and injected on the column which then is eluted with a gradient of 40-60% CH₃CN in 0.1% TFA 10 ml/min during 50 min at 40° C. The peptide containing fractions is collected. The purified peptide is lyophilized after dilution of the eluate with water.

The final product obtained may be characterised by analytical RP-HPLC (retention time) and by LCMS.

The RP-HPLC analysis performed in these in the experimental section was performed using UV detection at 214 nm and a Vydac 218TP54 4.6 mm×250 mm 5μ C-18 silica column (The Separations Group, Hesperia, USA) which was eluted at 1 ml/min at 42° C. The different elution conditions were:
A1: Equilibration of the column with in a buffer consisting of 0.1M (NH₄)₂SO₄, which was adjusted to pH 2.5 with concentrated H₂SO₄ and elution by a gradient of 0% to 60% CH₃CN in the same buffer during 50 min.
B1: Equilibration of the column with 0.1% TFA/H₂O and elution by a gradient of 0% CH₃CN/0.1% TFA/H₂O to 60% CH₃CN/0.1% TFA/H₂O during 50 min.
B6: Equilibration of the column with 0.1% TFA/H₂O and elution by a gradient of 0% CH₃CN/0.1% TFA/H₂O to 90% CH₃CN/0.1% TFA/H₂O during 50 min.
An alternative system was:
B4: The RP-analyses was performed using a Alliance Waters 2695 system fitted with a Waters 2487 dualband detector. UV detections at 214 nm and 254 nm were collected using a Symmetry300 C18, 5 um, 3.9 mm×150 mm column, 42° C. Eluted with a linear gradient of 5-95% acetonitrile, 90-0% water, and 5% trifluoroacetic acid (1.0%) in water over 15 minutes at a flow-rate of 1.0 min/min.

### LCMS Method 1:

LCMS was performed on a setup consisting of Hewlett Packard series 1100 G1312A Bin Pump, Hewlett Packard series 1100 Column compartment, Hewlett Packard series 1100 G1315A DAD diode array detector, Hewlett Packard series 1100 MSD and Sedere 75 Evaporative Light Scattering detector controlled by HP Chemstation software. The HPLC pump is connected to two eluent reservoirs containing:

### A: 0.05% TFA/water

### B: 0.05% TFA/acetonitrile

Or alternatively the two systems may be:

A: 10 mM NH₄OH in water
B: 10 mM NH₄OH in 90% acetonitrile

The analysis was performed at 23° C. by injecting an appropriate volume of the sample (preferably 20 μl) onto the column which is eluted with a gradient of A and B.

The HPLC conditions, detector settings and mass spectrometer settings used are giving in the following table.

| | |
|---|---|
| Column | Waters Xterra MS C-18 (50 × 3 mm id 5 μm) |
| Gradient | 5%-100% acetonitrile linear during 6.5 min at 1.5 ml/min |
| Detection | 210 nm (analogue output from DAD) |

ELS (analogue output from ELS)
MS ionisation mode API-ES. Scan 550-1500 amu step 0.1 amu

### LCMS Method 2:

Alternatively, LC-MS analysis could be performed on a PE-Sciex API 100 mass spectrometer equipped with two Perkin Elmer Series 200 Micropumps, a Perkin Elmer Series 200 autosampler, a Applied Biosystems 785A UV detector and a Sedex 75 Evaporative Light scattering detector. A Waters Xterra 3.0 mm×50 mm 5μ C-18 silica column was eluted at 1.5 ml/min at room temperature. It was equilibrated with 5% CH₃CN/0.05% TFA/H₂O and eluted for 1.0 min with 5% CH₃CN/0.05% TFA/H₂O and then with a linear gradient to 90% CH₃CN/0.05% TFA/H₂O over 7 min. Detection was by UV detection at 214 nm and Evaporative light Scattering. A fraction of the column eluate was introduced into the ionspray interface of a PE-Sciex API 100 mass spectrometer. The mass range 300-2000 amu was scanned every 2 seconds during the run.

### LCMS Method 3:

Alternatively the LC-MS analysis was performed on a XTerra MS C₁₈ 5 μl 3.0×50 mm column (Waters, Milford Mass., USA) which is eluted at 1 ml/min at room temperature. The HPLC system was equipped with a Sciex API 150 mass spectrometer scanning from 200-1500 amu every 2 seconds of the run.

### Gradient:

| | Total Time | Flow Rate | ACN 0.08% | MQ 0.1% |
|---|---|---|---|---|
| Step | (min) | (μl/min) | TFA (%) | TFA (%) |
| 0 | 1.00 | 1500 | 5.0 | 95.0 |
| 1 | 3.00 | 1500 | 20.0 | 80.0 |
| 2 | 16.00 | 1500 | 50.0 | 50.0 |
| 3 | 18.00 | 1500 | 90.0 | 10.0 |
| 4 | 18.10 | 1500 | 5.0 | 95.0 |
| 5 | 20.00 | 1500 | 5.0 | 95.0 |
| 6 | 22.00 | 0 | 0.0 | 100.0 |

MALDI-TOF MS analysis was carried out using a Voyager RP instrument (PerSeptive Biosystems Inc., Framingham, Mass.) equipped with delayed extraction and operated in linear mode. Alpha-cyano-4-hydroxy-cinnamic acid was used as matrix, and mass assignments were based on external calibration.

### Radioligand Binding to Plasma Membranes Expressing the Human GLP-1 Receptor

The binding assay was performed with purified plasma membranes containing the human GLP-1 receptor. The plasma membranes containing the receptors were purified from stably expressing BHK tk-ts 13 cells. The membranes were diluted in Assay Buffer (50 mM HEPES, 5 mM EGTA, 5 mM MgCl₂, 0.005% Tween 20, pH=7.4) to a final concentration of 0.2 mg/ml of protein and destributed to 96-well microtiter plates precoated with 0.3% PEI. Membranes in the presence of 0.05 nM [¹²⁵I]GLP-1, unlabelled ligands in increasing concentrations and different HSA concentrations (0.005%, 0.05%, and 2%) were incubated 2 hr at 30° C. After incubation, unbound ligands were separated from bound ligands by filtration through a vacuum-manifold followed by 2×100 μl washing with ice cold assay buffer. The filters were dried overnight at RT, punched out and quantified in a γ-counter.

### Example 1

### O,O′-Bis-(2-((Arg³⁴,Lys²⁶-GLP-1(7-37)-N^{epsilon,26}yl)carbonyl)ethyl)tetraethyleneglycol

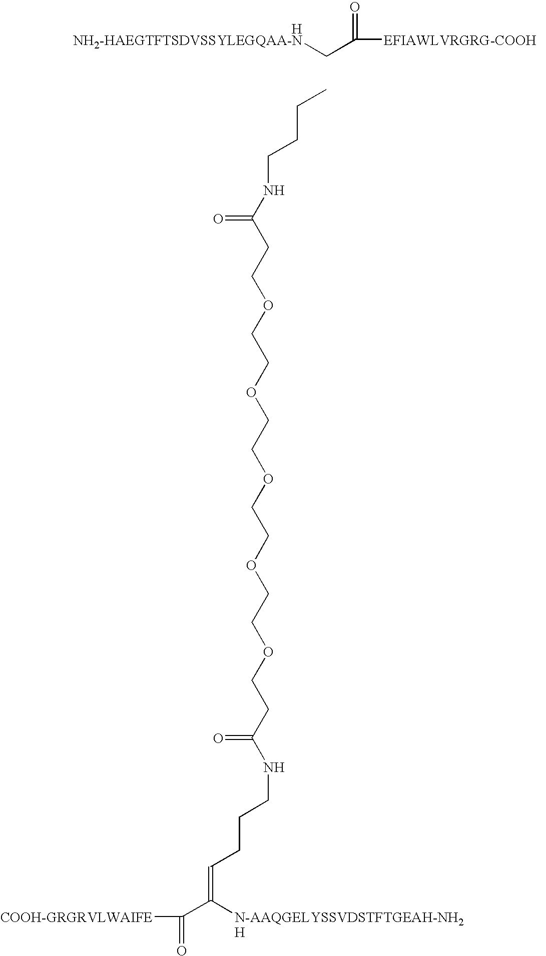

### Dimerization:

Arg³⁴GLP-1(7-37) was expressed in yeast (S. cerevisiae) by conventional recombinant technology as described elsewhere (WO 98/08871). Arg³⁴GLP-1(7-37) in the fermentation broth was then purified by conventional reversed phase chromatography and subsequently precipitated at the isoelectric pH of the peptide, i.e. at pH 5.4. Dimerization was performed using 1412 mg of the isoprecipitate containing approximately 470 mg of monomeric Arg³⁴GLP-1(7-37) peptide based upon the absorbance at 280 nm at neutral pH using a 1 cm cell. Molar extinction coefficient of Trp 5560 AU/mmol/ml, Tyr 1200 AU/mmol/ml. The amount of peptide in mg peptide pr mL was calculated as mg/mL=(A280×DF×MF)/e. A280 is the actual absorbance of the solution at 280 nm i a 1-cm cell. MW is molecular weight of the peptide, DF the dilution factor relative to the stock solution and e is the combined molar extintion coefficient of each of the Trp or Tyr chromophores at 280 nm. E will in this case be e=1×5560 AU/mmol/ml+0×1200 AU/mmol/ml totaling 5560 AU/mmol/ml.

72 mg Bis-dPEG₆™ NHS ester from Quanta biodesign (QBD product number 10224)

The peptide was taken up in 4 mL DMSO+10 mL H2O. 230 microL DIPEA was added followed by 72 mg Bis-dPEG₆™ NHS ester from Quanta biodesign (QBD product number 10224). The reaction was run overnight). Yielding the product in 83.5% purity on the LC/MS method peptid1500_{—}20.RT:11.84 ms:1414.9 (M+5/5).

The peptide was injected directly on a Gilson semi prep system (2 cm column, gradient 16-46% ACN) Yield after freeze drying 132 mg.

LCMS method 3 (peptid 1500_{—}20) RT:11.82 ms: msfound. 1415.0 (M+5/5).

### Example 2

### O,O′-Bis-(2-((Arg¹², Leu¹⁴, Arg²⁷, Lys³⁴-Exendin-4 (1-39) N^{epsilon,34}yl)carbonyl)ethyl)-tetraethyleneglycol

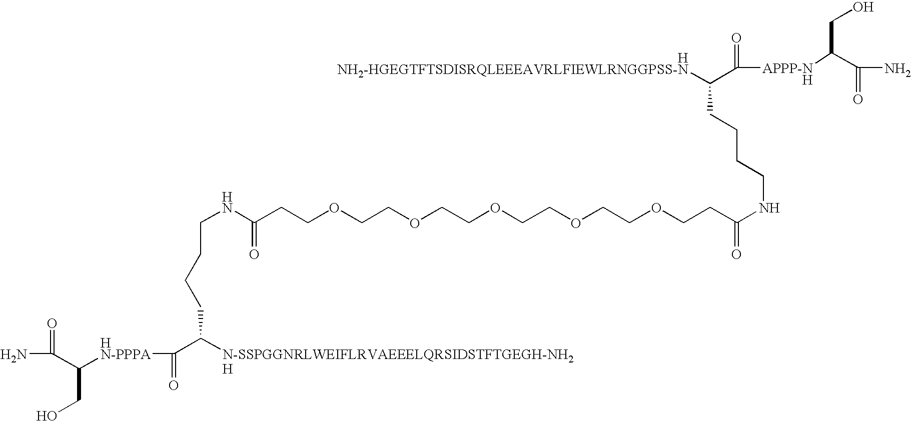

A resin (Rink amide, 0.68 mmol/g Novabiochem 0.25 mmole) was used to produce the primary sequence on an ABI433A machine according to manufacturers guidelines. All protecting groups were acid labile.

### Procedure

The above prepared resin (0.25 mmole) containing the GLP-1 analogue amino acid sequence was cleaved from the resin by stirring for 180 min at room temperature with a mixture of trifluoroacetic acid, water and triisopropylsilane (95:2.5:2.5 15 ml). The cleavage mixture was filtered and the filtrate was concentrated to an oil in vaccuum. The crude peptide was precipitated from this oil with 45 ml diethyl ether and washed 3 times with 45 ml diethyl ether. The crude peptide was purified by preparative HPLC on a 20 mm×250 mm column packed with 7μ C-18 silica. The crude peptide was dissolved in 5 ml 50% acetic acid in water and diluted to 20 ml with H₂O and injected on the column which then was eluted with a gradient of 40-60% (CH₃CN in water with 0.1% TFA) 10 ml/min during 50 min at 40° C. The peptide containing fractions were collected. The purified peptide was lyophilized after dilution of the eluate with water.

HPLC: (method B6): RT=28.582 min

LCMS: RT=11.29 m/z=1432.9 (M+3H)³⁺.

The peptide was dimerized according to the procedure described for the yeast extract in example 1.

LCMS method 3: RT=12.91, m/z=1483.1 (M+6H)⁶⁺, 1271.6 (M+7H)⁷⁺, 1112.7 (M+8H)⁸⁺, 989.2 (M+9H)⁹⁺.

HPLC: (method B6): RT=30.1 min, m/z=8894.0 (MALDI-TOF, Sinapinic acid matrix)

### Example 3

### O,O′-Bis-(2-((Leu¹⁴, Arg²⁷-Exendin-4 (1-39)-N^{epsilon,12}yl)carbonyl)ethyl)octaethyleneglycol

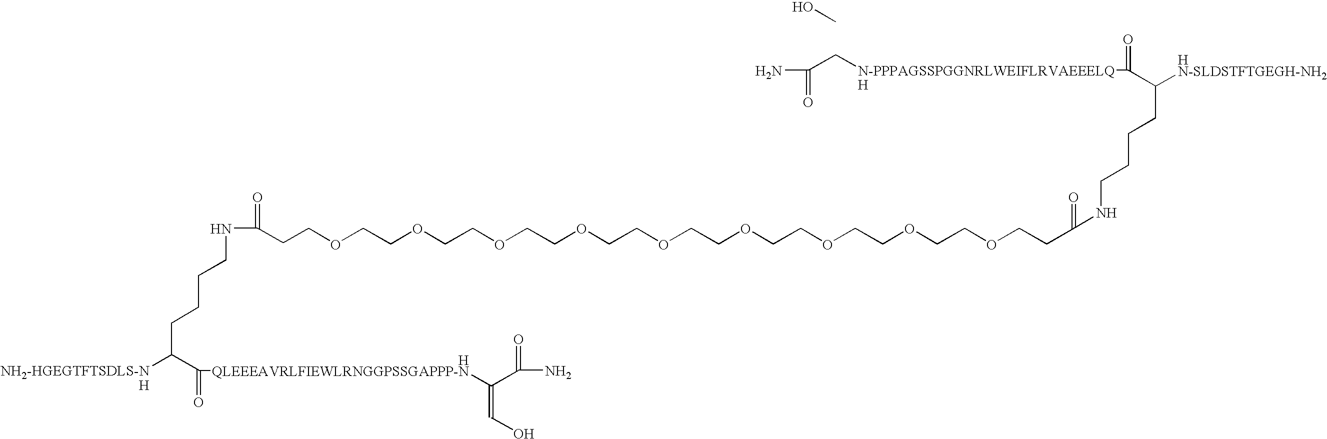

Synthesized according to procedure described in example 1 and 2. Bis-dPEG₉^{M} NHS ester from Quanta biodesign (QBD product number 10246)

HPLC: (method B6): RT=29.8 min, m/z=8871.3 (MALDI-TOF, Sinapinic acid matrix)

### Example 4

### O,O′-Bis-(2-((Leu¹⁴, Arg²⁷-Exendin-4 (1-39)-N^{epsilon,12}yl)carbonyl)ethyl)tetraethyleneglycol

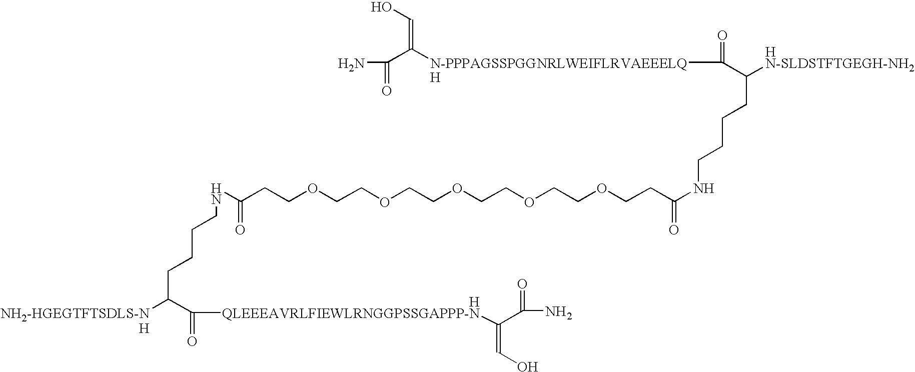

Synthesized according to procedure described in example 1 and 2

HPLC: (method B6): RT=29.5 min, m/z=8695.6 (MALDI-TOF, Sinapinic acid matrix)

### Example 5

### O,O′-Bis-(2-((Arg¹², Leu¹⁴, Arg²⁷, Lys³⁴-Exendin-4 (1-39) N^{epsilon,34}yl) carbonyl)ethyl)-octaethyleneglycol

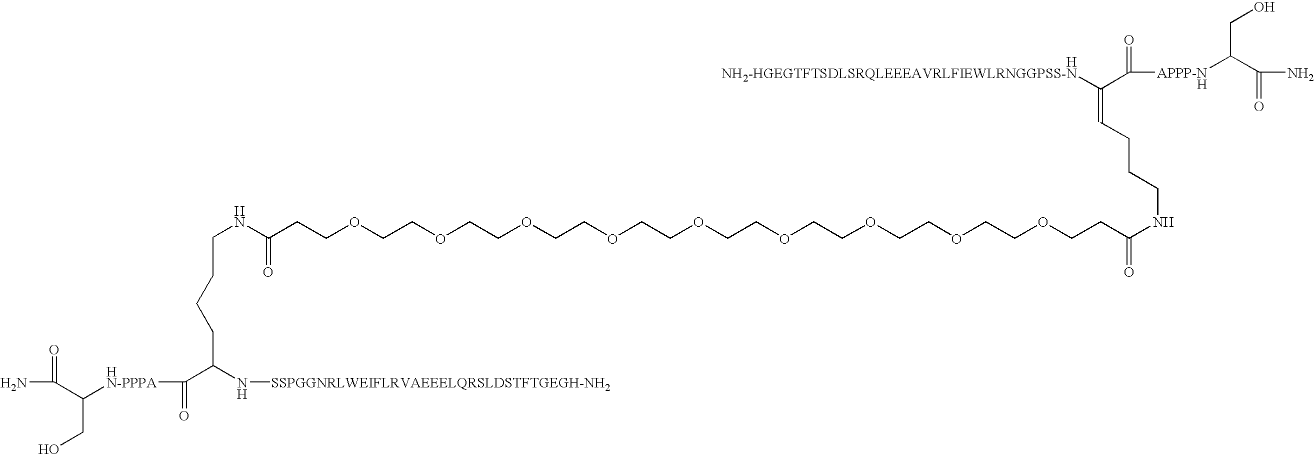

Synthesized according to procedure described in example 1 and 2

HPLC: (method B6): RT=30.1 min, m/z=9070.1 (MALDI-TOF, Sinapinic acid matrix)

### Example 6

### O,O′-Bis-(2-((Arg¹², Leu¹⁴, Lys²⁰, Arg²⁷-Exendin-4 (1-39) N^{epsilon,20}yl) carbonyl)ethyl)-tetraethyleneglycol

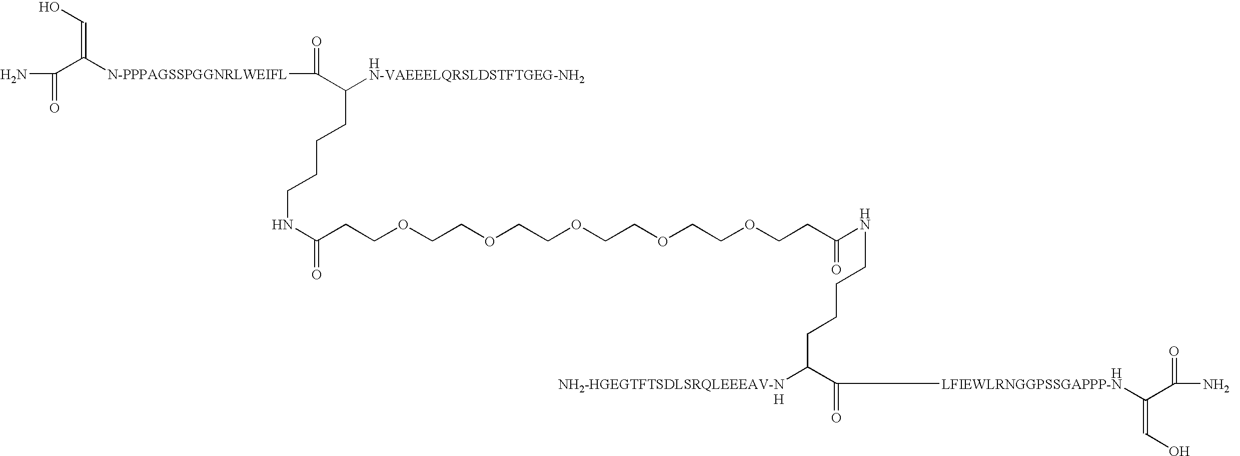

Synthesized according to procedure described in example 1 and 2

LCMS method 3: RT=11.73 min, m/z=8695.6 (MALDI-TOF, Sinapinic acid matrix)

### Example 7

### O,O′-Bis-(2-((Arg¹², Leu¹⁴, Lys²⁰, Arg²⁷-Exendin-4 (1-39) N^{epsilon,20}yl)carbonyl)ethyl)-octaethyleneglycol

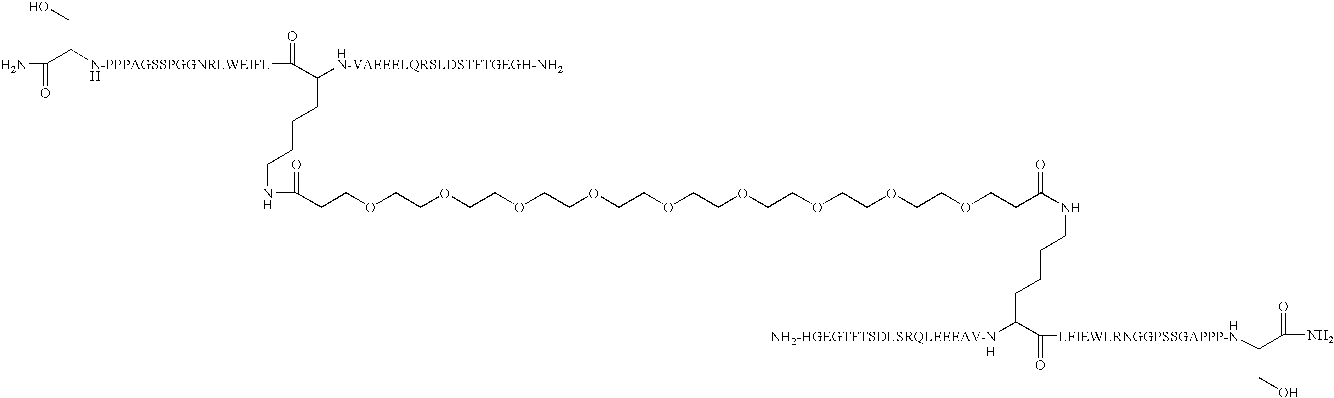

Synthesized according to procedure described in example 1 and 2

LCMS method 3: RT=11.77 min, m/z=8871.8 (MALDI-TOF, Sinapinic acid matrix)

### Example 8

### O,O′-Bis-(2-((Arg¹², Leu¹⁴-Exendin-4 (1-39) N^{epsilon,27}yl)carbonyl)ethyl)-octaethyleneglycol

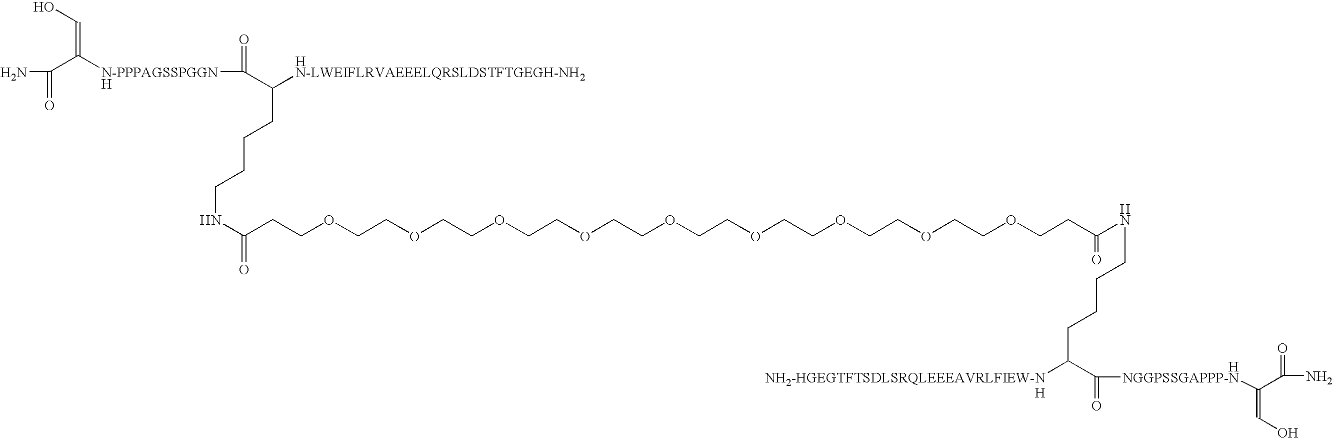

Synthesized according to procedure described in example 1 and 2

HPLC: (method B6): RT=30.5 min, m/z=8870.3 (MALDI-TOF, Sinapinic acid matrix)

### Example 9

### O,O′-Bis-(2-((Arg¹², Leu¹⁴-Exendin-4 (1-39) N^{epsilon,27}yl)carbonyl)ethyl)-tetraethyleneglycol

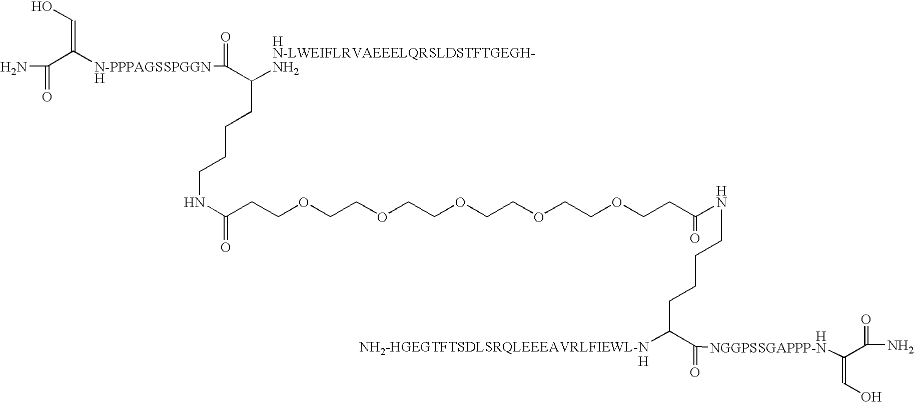

Synthesized according to procedure described in example 1 and 2

HPLC: (method B6): RT=30.6 min m/z=8695.6 (MALDI-TOF, Sinapinic acid matrix)

### Example 10

### N,N′-bis((((S)-5-([Aib,8,22,35]GLP-1(1-37)yl)-5-carbamoylpentyl)carbamoyl)methoxy)hexan-1,6-diimine

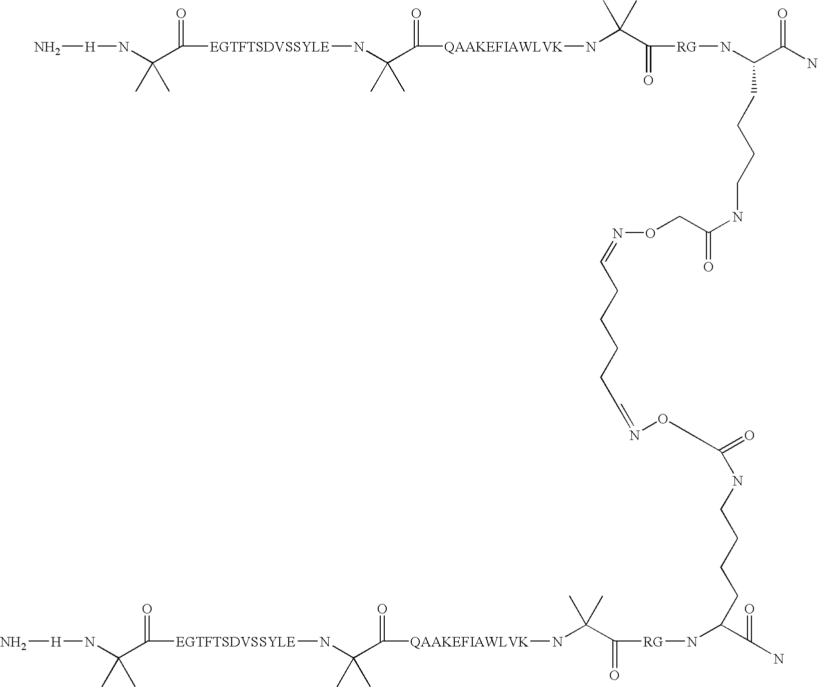

### Preparation of GLP-1 Containing an Oxyamino Group Attached to the Side-Chain of Lys

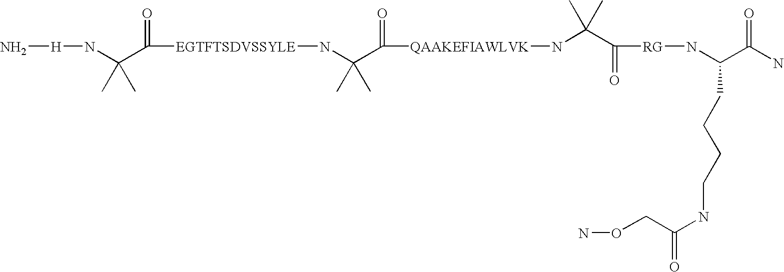

[Aib8,22,35]GLP-1(1-37)Lys(2-aminoxy acetyl) amide

The peptide was prepared on Rink amide Tentagel (0.22 mmol/g, 450 mg) using a standard Fmoc-chemistry protocol (4 eq. AA, 4 eq. DIC and 4 eq. HOAt and 25% pip in NMP to remove the Fmoc-group). The Lys residue was side-chain protected as Lys(Dde) and the oxyamino group was first introduced to the C-terminal at the side-chain of Lys by removed the Dde group with 2% TFA and 2% TIS in DCM and then coupled Boc-NH—O—CH₂—CO₂H. The peptide sequence was generated on the Apex348 from Advanced Chemtech. The peptide was finally cleaved with 95% TFA (aq) and TIS. Then, the peptide was characterized by LC-MS and isolated by preparative HPLC using the gradient of 30% to 70% buffer B over 50 min.

LC-MS: 3625.3. Calculated for C₁₆₄H₂₅₄N₄₄O₄₉: 3626.1

### Dimerization of GLP-1 Using Oxime Ligation.

The peptide [Aib8,22,35]GLP-1(7-37)Lys(CO—CH₂—ONH₂) (2.2 μmol) was added to 90% DMSO (aq) (30 μl) containing CHO—(CH₂)₄—CHO (0.5 μmol) and pH was adjusted to 5 with NaOAc. The solution was stirred at 28° C. for 2 days and the progress of the reaction was monitored by LC-MS. The product was finally isolated by preparative HPLC using a gradient of 30% to 70% buffer B over 50 min.

LC-MS: 7340.3. Calculated for C₃₃₄H₅₁₄N₈₈O₉₈: 7330.4

### Example 11

### N,N′-bis((((S)-5-(N-epsilon26[2-(2-[2-(2-[2-(2[4-(17-carboxyheptadecanoylamino)-4(S)-carboxybutyrylamino]ethoxy)ethoxy]acetylamino)ethoxy]ethoxy)acetyl][Aib8,Arg34]GLP-1(7-37)yl)-5-carbamoylpentyl)carbamoyl)methoxy)ethan-1,2-diimine

Synthesis of GLP-1 Containing Both an Oxyamino Group and a Protracted moiety at its Side-Chain of Lys.

### N-epsilon26[2-(2-[2-(2-[2-(2[4-(17-carboxyheptadecanoylamino)-4(S)-carboxybutyrylamino]ethoxy)ethoxy]acetylamino)ethoxy]ethoxy)acetyl][Aib8,Arg34]GLP-1(7-37)Lys(2-aminoxy acetyl)

The peptide was prepared on Rink amide Tentagel (0.22 mmol/g, 1 g, 0.22 mmol) using a standard Fmoc-chemistry protocol as described above. The oxyamino group was first introduced to the C-terminal at the side-chain of Lys by coupled Fmoc-Lys(Mtt) to the resin, removed the Mtt group with 2% TFA and 2% TIS in DCM and coupled Boc-NH—O—CH₂—CO₂H to the Lys side-chain. The entire peptide sequence was then generated on an Advanced Chemtech 348 synthesizer. In order to attach the protracted moiety into the sequence was Fmoc-Lys(Mtt) was applied in the synthesis. To the side-chain of Lys was coupled two units of OEG, γ-Glu and octadecanedioic acid using DIC and HOAt (3 equiv). The peptide was deprotected and cleaved from the resin with TFA/TIS/H₂O/thioanisol (90/5/3/2) and characterized by analytical HPLC and MALDI-MS. Finally was the peptide was purified by preparative HPLC using a gradient of 30% to 70% buffer B over 50 min.

HPLC: (method: 5% to 95% buffer B over 15 min and 95% buffer B for 5 min): RT=17.8 min.

MALDI-MS: 4314.3. Calculated for C₁₉₅H₃₀₆N₄₈O₆₂: 4314.9

Dimerization according to method described in example 11 LC-MS: 1731 (MH₅⁵⁺) Calculated for (MH₅⁵⁺): 1730

HPLC: (method: 10% to 90% buffer B over 25 min): RT=20.42 min.

1. A compound which comprises two glucagon-like peptide 1 (GLP-1) agonists linked to each other via a bifunctional cross-linker.

2. A compound according to claim 1, wherein the two GLP-1 agonists are identical.

3. A compound according to claim 2, wherein the two GLP-1 agonists are linked to the bifunctional crosslinker on the same amino acid residue.

4. A compound according to claim 1, wherein said GLP-1 agonists are GLP-1 or an analogue thereof.

5. A compound according to claim 1, wherein said GLP-1 agonists are exendin-4 or an analogue thereof.

6. A compound according to claim 1, which comprises two GLP-1 agonists linked via a bifunctional hydrophilic spacer W—(CH₂)ₗD[(CH₂)ₙE]ₘ(CH₂)ₚQ_{q}-, wherein
l, m and n independently are 1-20 and p is 0-10,
Q is -Z-(CH₂)ₗD[(CH₂)ₙG]ₘ(CH₂)ₚ—,
W is —(CH₂)ₚ[(CH₂)ₙG]ₘD(CH₂)ₗ-Z-,
q is an integer in the range from 0 to 5,
each D, E, and G independently are selected from —O—, —NR³—, —N(COR⁴)—, —PR⁵(O)—, and —P(OR⁶)(O)—, wherein R³, R⁴, R⁵, and R⁶ independently represent hydrogen or C₁₋₆-alkyl,
Z is selected from —C(O)NH—, —C(O)NHCH₂—, —OC(O)NH—, —C(O)NHCH₂CH₂—, —C(O)CH₂—, —C(O)CH═CH—, —(CH₂)ₛ—, —C(O)—, —C(O)O— or —NHC(O)—, wherein s is 0 or 1.

7. A compound according to claim 6, which has formula (I):
“GLP-1 compound”-Y—B—Y-“GLP-1 compound*” (I)
wherein
B is a hydrophilic spacer being W_{q}—(CH₂)ₗD[(CH₂)ₙE]ₘ(CH₂)ₚQ_{q}-, wherein
l, m and n independently are 1-20 and p is 0-10,
Q is -Z-(CH₂)ₗD [(CH₂)ₙG]ₘ(CH₂)ₚ—,
W is —(CH₂)ₚ[(CH₂)ₙG]ₘD(CH₂)ₗ-Z-,
q is an integer in the range from 0 to 5,
each D, E, and G independently are selected from —O—, —NR³—, —N(COR⁴)—, —PR⁵(O)—, and —P(OR⁶)(O)—, wherein R³, R⁴, R⁵, and R⁶ independently represent hydrogen or C₁₋₆-alkyl,
Z is selected from —C(O)NH—, —C(O)NHCH₂—, —OC(O)NH—, —C(O)NHCH₂CH₂—, —C(O)CH₂—, —C(O)CH═CH—, —(CH₂)ₛ—, —C(O)—, —C(O)O— or —NHC(O)—, wherein s is 0 or 1,
Y is a chemical group linking B and the GLP-1 agonist,
“GLP-1 compound” and “GLP-1 compound*” are GLP-1 agonists.

8. A compound according to claim 6, which has formula (II)
“GLP-1 compound”-Y—B—B′—Y′-“GLP-1 compound*” (II)
wherein
B and B′ are hydrophilic spacers independently selected from —W_{q}—(CH₂)ₗD[(CH₂)ₙE]ₘ(CH₂)ₚ-Q_{q}-, wherein
l, m and n independently are 1-20 and p is 0-10,
Q is -Z-(CH₂)ₗD [(CH₂)ₙG]ₘ(CH₂)ₚ—,
W is —(CH₂)ₚ[(CH₂)ₙG]ₘD(CH₂)ₗ-Z-
q is an integer in the range from 0 to 5,
each D, E, and G independently are selected from —O—, —NR³—, —N(COR⁴)—, —PR⁵(O)—, and —P(OR⁶)(O)—, wherein R³, R⁴, R⁵, and R⁶ independently represent hydrogen or C₁₋₆-alkyl,
Z is selected from —C(O)NH—, —C(O)NHCH₂—, —OC(O)NH—, —C(O)NHCH₂CH₂—, —C(O)CH₂—, —C(O)CH═CH—, —(CH₂)ₛ—, —C(O)—, —C(O)O— or —NHC(O)—, wherein s is 0 or 1,
Y is a chemical group linking B and the GLP-1 agonist, and
Y′ is a chemical group linking B′ and the GLP-1 agonist, and
“GLP-1 compound” and “GLP-1 compound*” are GLP-1 agonists.

9. A compound according to claim 8, wherein Y′ and Y′ is selected from the group consisting of —C(O)NH—, —NHC(O)—, —C(O)NHCH₂—, —CH₂NHC(O)—, —OC(O)NH—, —NHC(O)O—, —C(O)NHCH₂—, CH₂NHC(O)—, —C(O)CH₂—, —CH₂C(O)—, —C(O)CH═CH—, —CH═CHC(O)—, —(CH₂)ₛ—, —C(O)—, —C(O)O—, —OC(O)—, —NHC(O)— and —C(O)NH—, wherein s is 0 or 1.

10. A compound according to claim 6, wherein l is 1 or 2, n and m are independently 1-10 and p is 0-10.

11. A compound according to claim 6, wherein D is —O—.

12. A compound according to claim 6, wherein E is —O—.

13. A compound according to claim 6, wherein the hydrophilic spacer is
—CH₂O[(CH₂)₂O]ₘ(CH₂)ₚQ_{q}-, where m is 1-10, p is 1-3, and Q is -Z-
CH₂O[(CH₂)₂O]ₘ(CH₂)ₚ—.

14. A compound according to claim 6, wherein q is 0.

15. A compound according to claim 6, wherein q is 1.

16. A compound according to claim 6, wherein G is —O—.

17. A compound according to claim 6, wherein Z is selected from the group consisting of —C(O)NH—, —C(O)NHCH₂—, and —OC(O)NH—.

18. A compound according to claim 6, wherein l is 2.

19. A compound according to claim 6, wherein n is 2.

20. A compound according to claim 6, wherein the hydrophilic spacer B is —[CH₂CH₂O]ₘ₊₁(CH₂)ₚQ_{q}-.

21. A compound according to claim 1, wherein the GLP-1 compound comprises the an amino acid sequence of formula I:
Formula (I)
(SEQ. ID No: 1)
Xaa₁-Xaa₂-His-Gly-Xaa₅-Phe-Xaa₇-Xaa₈-Xaa₉-Xaa₁₀-
Xaa₁₁-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Phe-Xaa₂₃-Xaa₂₄-Trp-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉-Xaa₄₀-Xaa₄₁-Xaa₄₂
wherein
Xaa₁ is L-histidine, D-histidine, desamino-histidine, 2-amino-3-(2-aminoimidazol-4-yl)propionic acid, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine; or L-tyrosine
Xaa₂ is Ala, Gly, Val, Leu, Ile, Lys, Aib, 1-aminocyclopropanecarboxylic acid, 1-aminocyclobutanecarboxylic acid, 1-aminocyclopentanecarboxylic acid, 1-aminocyclohexanecarboxylic acid, 1-aminocycloheptanecarboxylic acid, or 1-aminocyclooctanecarboxylic acid;
Xaa₅ is Thr or Ser;
Xaa₇ is Thr or Ser;
Xaa₈ is Ser or Asp;
Xaa₉ is Glu or Asp;
Xaa₁₀ is Val, Met, Leu or Tyr;
Xaa₁₁ is Ser, or Asn;
Xaa₁₂ is Ser, Thr, Lys or Ile;
Xaa₁₃ is Tyr, Ile, Ala or Gln;
Xaa₁₄ is Leu or Met;
Xaa₁₅ is Asp or Glu;
Xaa₁₆ is Gly, Asn, Glu or Lys;
Xaa₁₇ is Leu, Gln, Glu or Ile;
Xaa₁₈ is Ala or His;
Xaa₁₉ is Ala, Gln or Val;
Xaa₂₀ is Lys, Arg or Gln;
Xaa₂₁ is Asp, Glu or Leu;
Xaa₂₃ is Ile or Val;
Xaa₂₄ is Ala, Asn or Glu;
Xaa₂₆ is Leu or Ile;
Xaa₂₇ is Val, Ile, Leu, Arg or Lys;
Xaa₂₈ is Lys, Gln, Ala or Asn;
Xaa₂₉ is Gly, Thr or Gln;
Xaa₃₀ is Arg, Lys or Gly;
Xaa₃₁ is Ile, Gly, Pro, amide or is absent;
Xaa₃₂ is Thr, Lys, Ser, amide or is absent;
Xaa₃₃ is Asp, Lys, Ser, amide or is absent;
Xaa₃₄ is Arg, Asn, Gly, amide or is absent;
Xaa₃₅ is Asp, Ala, amide or is absent;
Xaa₃₆ is Trp, Pro, amide or is absent;
Xaa₃₇ is Lys, Pro, amide or is absent;
Xaa₃₈ is His, Pro, amide or is absent;
Xaa₃₉ is Asn, Ser, amide or is absent;
Xaa₄₀ is Ile, amide or is absent;
Xaa₄₁ is Thr, amide or is absent;
Xaa₄₂ is Gln, amide or is absent;
provided that if Xaa₃₁, Xaa₃₂, Xaa₃₃, Xaa₃₄, Xaa₃₅, Xaa₃₆, Xaa₃₇, Xaa₃₈, Xaa₃₉, Xaa₄₀, Xaa₄₁, or Xaa₄₂ is absent then each amino acid residue downstream is also absent.

22. The compound according to claim 21, wherein the amino acid sequence is according to formula 2:
Formula (2)
(SEQ. ID No: 2)
His-Xaa₂-His-Gly-Xaa₅-Phe-Xaa₇-Xaa₈-Xaa₉-Xaa₁₀-
Xaa₁₁-Xaa₁₂-Xaa₁₃-Xaa₁₄-Xaa₁₅-Xaa₁₆-Xaa₁₇-Ala-
Xaa₁₉-Xaa₂₀-Xaa₂₁-Phe-Ile-Xaa₂₄-Trp-Leu-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉
wherein
Xaa₂ is Ala, Gly, Val, Leu, Ile, Lys, Aib, 1-aminocyclopropanecarboxylic acid, 1-aminocyclobutanecarboxylic acid, 1-aminocyclopentanecarboxylic acid, 1-aminocyclohexanecarboxylic acid, 1-aminocycloheptanecarboxylic acid, or 1-aminocyclooctanecarboxylic acid;
Xaa₅ is Thr or Ser;
Xaa₇ is Thr or Ser;
Xaa₈ is Ser or Asp;
Xaa₉ is Glu or Asp;
Xaa₁₀ is Val, Met, or Leu;
Xaa₁₁ is Ser or Asn;
Xaa₁₂ is Ser, Thr or Lys;
Xaa₁₃ is Tyr, Ile or Gln;
Xaa₁₄ is Leu or Met;
Xaa₁₅ is Asp or Glu;
Xaa₁₆ is Gly, Asn or Glu;
Xaa₁₇ is Leu, Gln or Glu;
Xaa₁₉ is Ala or Val;
Xaa₂₀ is Lys or Arg;
Xaa₂₁ is Asp, Glu or Leu;
Xaa₂₄ is Ala, Asn or Glu;
Xaa₂₇ is Val, Ile or Lys;
Xaa₂₈ is Lys, Gln or Asn;
Xaa₂₉ is Gly or Thr;
Xaa₃₀ is Arg, Lys or Gly;
Xaa₃₁ is Ile, Pro, amide or is absent;
Xaa₃₂ is Thr, Ser, amide or is absent;
Xaa₃₃ is Asp, Ser, amide or is absent;
Xaa₃₄ is Arg, Gly, amide or is absent;
Xaa₃₅ is Ala, amide or is absent;
Xaa₃₆ is Pro, amide or is absent;
Xaa₃₇ is Pro, amide or is absent;
Xaa₃₈ is Pro, amide or is absent;
Xaa₃₉ is Ser, amide or is absent;
provided that if Xaa₃₁, Xaa₃₂, Xaa₃₃, Xaa₃₄, Xaa₃₅, Xaa₃₆, Xaa₃₇, Xaa₃₈, or Xaa₃₉ is absent then each amino acid residue downstream is also absent.

23. The compound according to claim 21, wherein the amino acid sequence
is according to formula 3:
Formula (3)
(SEQ. ID No: 3)
His-Xaa₂-His-Gly-Thr-Phe-Thr-Ser-Asp-Xaa₁₀-Ser-
Xaa₁₂-Xaa₁₃-Xaa₁₄-Glu-Xaa₁₆-Xaa₁₇-Ala-Xaa₁₉-Xaa₂₀-
Xaa₂₁-Phe-Ile-Xaa₂₄-Trp-Leu-Xaa₂₇-Xaa₂₈-Gly-Xaa₃₀-
Xaa₃₁-Xaa₃₂-Xaa₃₃-Xaa₃₄-Xaa₃₅-Xaa₃₆-Xaa₃₇-Xaa₃₈-
Xaa₃₉
Xaa₂ is Ala, Gly, Val, Leu, Ile, Lys, Aib, 1-aminocyclopropanecarboxylic acid, 1-aminocyclobutanecarboxylic acid, 1-aminocyclopentanecarboxylic acid, 1-aminocyclohexanecarboxylic acid, 1-aminocycloheptanecarboxylic acid, or 1-aminocyclooctanecarboxylic acid;
Xaa₁₀ is Val or Leu;
Xaa₁₂ is Ser or Lys;
Xaa₁₃ is Tyr or Gln;
Xaa₁₄ is Leu or Met;
Xaa₁₆ is Gly or Glu;
Xaa₁₇ is Gln or Glu;
Xaa₁₉ is Ala or Val;
Xaa₂₀ is Lys or Arg;
Xaa₂₁ is Glu or Leu;
Xaa₂₄ is Ala or Glu;
Xaa₂₇ is Val or Lys;
Xaa₂₈ is Lys or Asn;
Xaa₃₀ is Arg, Lys or Gly;
Xaa₃₁ is Pro, amide or is absent;
Xaa₃₂ is Ser, amide or is absent;
Xaa₃₃ is Ser, amide or is absent;
Xaa₃₄ is Gly, amide or is absent;
Xaa₃₅ is Ala, amide or is absent;
Xaa₃₆ is Pro, amide or is absent;
Xaa₃₇ is Pro, amide or is absent;
Xaa₃₈ is Pro, amide or is absent;
Xaa₃₉ is Ser, amide or is absent;
provided that if Xaa₃₁, Xaa₃₂, Xaa₃₃, Xaa₃₄, Xaa₃₅, Xaa₃₆, Xaa₃₇, Xaa₃₈, or Xaa₃₉ is absent then each amino acid residue downstream is also absent.

24. A compound according to claim 1, wherein the two GLP-1 agonists are dimerised via an amino acid residue at one of the following positions:
GLP1: residue number 18, 22, 26, 34, 36, 37 or 38
Exendin-4: residue number 12, 16, 20, 27, 32, 33 or 34.

25. A compound according to claim 1, which is selected from
O,O′-Bis-(2-((Arg³⁴,Lys²⁶-GLP-1(7-37)-N^{epsilon,26}yl)carbonyl)ethyl)tetraethyleneglycol
O,O′-Bis-(2-((Gly⁸, Arg³⁴,Lys²⁶-GLP-1(7-37)-N^{epsilon,26}yl)carbonyl)ethyl)tetraethyleneglycol
O,O′-Bis-(2-((Aib⁸, Arg³⁴,Lys²⁶-GLP-1(7-37)-N^{epsilon,26}yl)carbonyl)ethyl)tetraethyleneglycol
O,O′-Bis-(2-((Val⁸, Arg³⁴,Lys²⁶-GLP-1(7-37)-N^{epsilon,26}yl)carbonyl)ethyl)tetraethyleneglycol
O,O′-Bis-(2-((Gly⁸, Arg²⁶, Arg³⁴, Lys²²-GLP-1(7-37)-N^{epsilon,22}yl)carbonyl)ethyl)tetraethyleneglycol
O,O′-Bis-(2-((Aib⁸, Arg²⁶, Arg³⁴, Lys²²-GLP-1(7-37)-N^{epsilon,22}yl)carbonyl)ethyl)tetraethyleneglycol
O,O′-Bis-(2-((Val⁸, Arg²⁶, Arg³⁴, Lys²²-GLP-1(7-37)-N^{epsilon,22}yl)carbonyl)ethyl)tetraethyleneglycol
O,O′-Bis-(2-((Gly⁸, Arg²⁶, Arg³⁴, Lys¹⁸-GLP-1(7-37)-N^{epsilon,18}yl)carbonyl)ethyl)tetraethyleneglycol
O,O′-Bis-(2-((Aib⁸, Arg²⁶, Arg³⁴, Lys¹⁸-GLP-1(7-37)-N^{epsilon,18}yl)carbonyl)ethyl)tetraethyleneglycol
O,O′-Bis-(2-((Val⁸, Arg²⁶, Arg³⁴, Lys¹⁸-GLP-1(7-37)-N^{epsilon,18}yl)carbonyl)ethyl)tetraethyleneglycol
O,O′-Bis-(2-((Gly⁸, Arg²⁶, Arg³⁴, Lys³⁴-GLP-1(7-37)-N^{epsilon,34}yl)carbonyl)ethyl)tetraethyleneglycol
O,O′-Bis-(2-((Aib⁸, Arg²⁶, Arg³⁴, Lys³⁴-GLP-1(7-37)-N^{epsilon,34}yl)carbonyl)ethyl)tetraethyleneglycol
O,O′-Bis-(2-((Val⁸, Arg²⁶, Arg³⁴, Lys³⁴-GLP-1(7-37)-N^{epsilon,34}yl)carbonyl)ethyl)tetraethyleneglycol
O,O′-Bis-(2-((Gly⁸, Arg²⁶, Arg³⁴, Lys³⁶-GLP-1(7-37)-N^{epsilon,36}yl)carbonyl)ethyl)tetraethyleneglycol
O,O′-Bis-(2-((Aib⁸, Arg²⁶, Arg³⁴, Lys³⁶-GLP-1(7-37)-N^{epsilon,36}yl)carbonyl)ethyl)tetraethyleneglycol
O,O′-Bis-(2-((Val⁸, Arg²⁶, Arg³⁴, Lys³⁶-GLP-1(7-37)-N^{epsilon,36}yl)carbonyl)ethyl)tetraethyleneglycol
O,O′-Bis-(2-((Gly⁸, Arg²⁶, Arg³⁴,Lys³⁷-GLP-1(7-37)-N^{epsilon,37}yl)carbonyl)ethyl)tetraethyleneglycol
O,O′-Bis-(2-((Aib⁸, Arg²⁶, Arg³⁴, Lys³⁷-GLP-1(7-37)-N^{epsilon,37}yl)carbonyl)ethyl)tetraethyleneglycol
O,O′-Bis-(2-((Val⁸, Arg²⁶, Arg³⁴, Lys³⁷-GLP-1(7-37)-N^{epsilon,37}yl)carbonyl)ethyl)tetraethyleneglycol
O,O′-Bis-(2-((Gly⁸, Arg²⁶, Arg³⁴-GLP-1(7-37)-Lys-N^{epsilon}yl)carbonyl)ethyl)tetraethylene-glycol
O,O′-Bis-(2-((Aib⁸, Arg²⁶, Arg³⁴-GLP-1(7-37)-Lys-N^{epsilon}yl)carbonyl)ethyl)tetraethylene-glycol
O,O′-Bis-(2-((Val⁸, Arg²⁶, Arg³⁴-GLP-1(7-37)-Lys-N^{epsilon}yl)carbonyl)ethyl)tetraethylene-glycol
O,O′-Bis-(2-((Arg³⁴,Lys²⁶GLP-1(7-37)-N^{epsilon,26}yl)carbonyl)ethyl)tetraethyleneglycol
O,O′-Bis-(2-((Gly⁸, Arg³⁴,Lys²⁶-GLP-1(7-37)-N^{epsilon,26}yl)carbonyl)ethyl)octaethyleneglycol
O,O′-Bis-(2-((Aib⁸, Arg³⁴,Lys²⁶-GLP-1(7-37)-N^{epsilon,26}yl)carbonyl)ethyl)octaethyleneglycol
O,O′-Bis-(2-((Val⁸, Arg³⁴,Lys²⁶-GLP-1(7-37)-N^{epsilon,26}yl)carbonyl)ethyl)octaethyleneglycol
O,O′-Bis-(2-((Gly⁸, Arg²⁶, Arg³⁴, Lys²²-GLP-1(7-37)-N^{epsilon,22}yl)carbonyl)ethyl)octaethylene-glycol
O,O′-Bis-(2-((Aib⁸, Arg²⁶, Arg³⁴, Lys²²-GLP-1(7-37)-N^{epsilon,22}yl)carbonyl)ethyl)octaethylene-glycol
O,O′-Bis-(2-((Val⁸, Arg²⁶, Arg³⁴, Lys²²-GLP-1(7-37)-N^{epsilon,22}yl)carbonyl)ethyl)octaethylene-glycol
O,O′-Bis-(2-((Gly⁸, Arg²⁶, Arg³⁴, Lys¹⁸-GLP-1(7-37)-N^{epsilon,18}yl)carbonyl)ethyl)octaethyleneglycol
O,O′-Bis-(2-((Aib⁸, Arg²⁶, Arg³⁴, Lys¹⁸-GLP-1(7-37)-N^{epsilon,18}yl)carbonyl)ethyl)octaethyleneglycol
O,O′-Bis-(2-((Val⁸, Arg²⁶, Arg³⁴, Lys¹⁸-GLP-1(7-37)-N^{epsilon,18}yl)carbonyl)ethyl)octaethylene-glycol
O,O′-Bis-(2-((Gly⁸, Arg²⁶, Arg³⁴, Lys³⁴-GLP-1(7-37)-N^{epsilon,34}yl)carbonyl)ethyl)octaethylene-glycol
O,O′-Bis-(2-((Aib⁸, Arg²⁶, Arg³⁴, Lys³⁴-GLP-1(7-37)-N^{epsilon,34}yl)carbonyl)ethyl)octaethylene-glycol
O,O′-Bis-(2-((Val⁸, Arg²⁶, Arg³⁴, Lys³⁴-GLP-1(7-37)-N^{epsilon,34}yl)carbonyl)ethyl)octaethylene-glycol
O,O′-Bis-(2-((Gly⁸, Arg²⁶, Arg³⁴, Lys³⁶-GLP-1(7-37)-N^{epsilon,36}yl)carbonyl)ethyl)octaethylene-glycol
O,O′-Bis-(2-((Aib⁸, Arg²⁶, Arg³⁴, Lys³⁶-GLP-1(7-37)-N^{epsilon,36}yl)carbonyl)ethyl)octaethylene-glycol
O,O′-Bis-(2-((Val⁸, Arg²⁶, Arg³⁴, Lys³⁶-GLP-1(7-37)-N^{epsilon,36}yl)carbonyl)ethyl)octaethylene-glycol
O,O′-Bis-(2-((Gly⁸, Arg²⁶, Arg³⁴,Lys³⁷-GLP-1(7-37)-N^{epsilon,37}yl)carbonyl)ethyl)octaethylene-glycol
O,O′-Bis-(2-((Aib⁸, Arg²⁶, Arg³⁴, Lys³⁷-GLP-1(7-37)-N^{epsilon,37}yl)carbonyl)ethyl)octaethylene-glycol
O,O′-Bis-(2-((Val⁸, Arg²⁶, Arg³⁴, Lys³⁷-GLP-1(7-37)-N^{epsilon,37}-yl)carbonyl)ethyl)octaethylene-glycol
O,O′-Bis-(2-((Gly⁸, Arg²⁶, Arg³⁴, GLP-1(7-37)-Lys-N^{epsilon}yl)carbonyl)ethyl)octaethyleneglycol
O,O′-Bis-(2-((Aib⁸, Arg²⁶, Arg³⁴-GLP-1(7-37)-Lys-N^{epsilon}yl)carbonyl)ethyl)octaethyleneglycol
O,O′-Bis-(2-((Val⁸, Arg²⁶, Arg³⁴-GLP-1(7-37)-Lys-N^{epsilon}yl)carbonyl)ethyl)octaethyleneglycol
O,O′-Bis-(2-((Arg¹², Leu¹⁴, Arg²⁷, Lys³⁴-Exendin-4 (1-39) N^{epsilon,34}yl)carbonyl)ethyl)-tetraethylene-glycol
O,O′-Bis-(2-((Leu¹⁴, Arg²⁷-Exendin-4 (1-39)-N^{epsilon,12}yl)carbonyl)ethyl)octaethyleneglycol
O,O′-Bis-(2-((Leu¹⁴, Arg²⁷-Exendin-4 (1-39)-N^{epsilon,12}yl)carbonyl)ethyl)tetraethylene-glycol
O,O′-Bis-(2-((Arg¹², Leu¹⁴, Arg²⁷, Lys³⁴-Exendin-4 (1-39) N^{epsilon,34}yl)carbonyl)ethyl)-octaethylene-glycol
O,O′-Bis-(2-((Arg¹², Leu¹⁴, Lys²⁰, Arg²⁷-Exendin-4 (1-39) N^{epsilon,20}yl)carbonyl)ethyl)-tetraethyleneglycol
O,O′-Bis-(2-((Arg¹², Leu¹⁴, Lys²⁰, Arg²⁷-Exendin-4 (1-39) N^{epsilon,20}yl)carbonyl)ethyl)-octaethyleneglycol
O,O′-Bis-(2-((Arg¹², Leu¹⁴-Exendin-4 (1-39) N^{epsilon,27}yl)carbonyl)ethyl)-octaethyleneglycol
O,O′-Bis-(2-((Arg¹², Leu¹⁴-Exendin-4 (1-39) N^{epsilon,27}yl) carbonyl)ethyl)-tetraethyleneglycol
N,N′-bis((((S)-5-([Aib,8,22,35]GLP-1(1-37)yl)-5-carbamoylpentyl)carbamoyl)methoxy)hexan-1,6-diimine
N,N′-bis((((S)-5-(N-epsilon26[2-(2-[2-(2-[2-(2 [4-(17-carboxyheptadecanoylamino)-4(S)-carboxybutyrylamino]ethoxy)ethoxy]acetylamino)ethoxy]ethoxy)acetyl][Aib8,Arg34]GLP-1(7-37)yl)-5-carbamoylpentyl)carbamoyl)methoxy)ethan-1,2-diimine

26. A method for increasing the pulmonal bioavailability in a patient of a GLP-1 agonist, characterised in dimerisation of said GLP-1 agonist via a bifunctional crosslinker so as to produce a compound according to claim 1.

27. A method for increasing the ratio of pulmonal bioavailability to potency in a patient of a GLP-1 agonist, said method comprising dimerising said GLP-1 agonist via a bifunctional crosslinker so as to produce a compound according to claim 1.

28. A pharmaceutical composition comprising a compound according to claim 1 and a pharmaceutically acceptable excipient.

29. The pharmaceutical composition according to claim 28 which is suited for pulmonal administration.

30-33. (canceled)

34. A compound according to claim 1, said compound further comprising a protracting moiety covalently attached to any or both strands of the dimeric GLP-1 agonists.

35. A compound according to claim 1, said compound further comprising a protracting moiety covalently attached to any or both strands of the dimeric GLP-1 agonists and the agonist is GLP-1 or analogues thereof.

36. A compound according to claim 1, said compound further comprising a protracting moiety covalently attached to any or both strands of the dimeric GLP-1 agonists and the agonist is Exendin-4 or analogues thereof.

37. A compound according to claim 34, where the protracting moiety is capable of binding to albumin.

38. A compound according to claim 34, where the protracting moiety is polyethyleneglycol.

39. A method for increasing the ratio of pulmonal bioavailability to potency in a patient of a GLP-1 agonist, said method comprising dimerising said GLP-1 agonist via a bifunctional crosslinker so as to produce a compound according to claim 34.

40. A pharmaceutical composition comprising a compound according to claim 34 and a pharmaceutically acceptable excipient.

41. The pharmaceutical composition according to claim 40 which is suited for pulmonal administration.

42-44. (canceled)

45. A method for treating hyperglycemia, type 2 diabetes, impaired glucose tolerance, type 1 diabetes, obesity, hypertension, syndrome X, dyslipidemia, cognitive disorders, atheroschlerosis, myocardial infarction, stroke, coronary heart disease and other cardiovascular disorders, inflammatory bowel syndrome, dyspepsia or gastric ulcers, said method comprising administering to a subject in need of said treatment an effective amount of a compound according to claim 1.

46. A method for delaying or preventing disease progression in type 2 diabetes in a subject, said method comprising administering to a subject in need of such treatment an effective amount of a compound according to claim 1.

47. A method for decreasing food intake, decreasing β-cell apoptosis, increasing β-cell function and β-cell mass, and/or for restoring glucose sensitivity to β-cells, said method comprising administering to a subject in need of such treatment an effective amount of a compound according to claim 1.

48. A method for treating hyperglycemia, type 2 diabetes, impaired glucose tolerance, type 1 diabetes, obesity, hypertension, syndrome X, dyslipidemia, cognitive disorders, atheroschlerosis, myocardial infarction, stroke, coronary heart disease and other cardiovascular disorders, inflammatory bowel syndrome, dyspepsia or gastric ulcers, said method comprising administering to a subject in need of said treatment an effective amount of a compound according to claim 34.

49. A method for delaying or preventing disease progression in type 2 diabetes in a subject, said method comprising administering to a subject in need of such treatment an effective amount of a compound according to claim 34.
